(19) United States
(12) Patent Application Publication
(10) Pub. No.: US 2020/0063194 A1
(43) Pub. Date: Feb. 27, 2020

(54) COMPREHENSIVE SINGLE MOLECULE ENHANCED DETECTION OF MODIFIED CYTOSINES

(71) Applicants: Jingyue Ju, Englewood Cliffs, NJ (US); Timothy H. Bestor, New York, NY (US); James J. Russo, New York, NY (US); Steffen Jockusch, New York, NY (US); Xiaoxu Li, New York, NY (US)

(72) Inventors: Jingyue Ju, Englewood Cliffs, NJ (US); Timothy H. Bestor, New York, NY (US); James J. Russo, New York, NY (US); Steffen Jockusch, New York, NY (US); Xiaoxu Li, New York, NY (US)

(73) Assignee: THE TRUSTEES OF COLUMBIA UNIVERSITY IN THE CITY OF NEW YORK, New York, NY (US)

(21) Appl. No.: 16/499,997

(22) PCT Filed: Apr. 3, 2018
(86) PCT No.: PCT/US18/25962

§ 371 Date: Oct. 1, 2019

Related U.S. Application Data
(60) Provisional application No. 62/481,017, filed on Apr. 3, 2017.
(60) Provisional application No. 62/487,360, filed on Apr. 19, 2017.
(60) Provisional application No. 62/534,549, filed on Jul. 19, 2017.

(51) Int. Cl. C12Q 1/6827; C12N 9/10

(52) U.S. Cl. CPC C12Q 1/6827; C12N 9/1051

(57) ABSTRACT

The subject invention provides a method of determining whether a cytosine at a predefined position within a single strand of a double-stranded DNA of known sequence is hydroxymethylated.

The invention also provides a method of determining whether a cytosine at a predefined position within a single strand of a double-stranded DNA of known sequence is unmethylated.

The invention further provides a method of determining whether a cytosine at a predefined position within a single strand of a double-stranded DNA of known sequence is methylated but not hydroxymethylated.

The invention also provides a method of determining whether a cytosine present at a predefined position within a single strand of a double-stranded DNA of known sequence, and within a CpG site, is unmethylated.

This application claims priority of U.S. Provisional Application Nos. 62/534,549, filed Jul. 19, 2017, 62/487,360, filed Apr. 19, 2017 and 62/481,017, filed Apr. 3, 2017, the content of each of which is hereby incorporated by reference in its entirety.

Throughout this application, various publications are referenced. Full citations for these references are present immediately before the claims. The disclosures of these publications in their entireties are hereby incorporated by reference into this application to more fully describe the state of the art to which this invention pertains.

### BACKGROUND OF THE INVENTION

Genomic methylation patterns are essential for cell viability, (Li 1992) and abnormal DNA methylation is an important factor in the etiology of ICF syndrome, fragile X syndrome, human cancer (reviewed in Goll 2005), some cases of Sotos syndrome (Lehman 2012), and hereditary sensorineural and dementia syndromes (Klein 2011). Cancer cells show strong and heterogenous abnormalities in genomic methylation patterns, with global losses and focal gains in DNA methylation thought to play an important role in cellular transformation (O'Donnell 2014). However, extant methods for methylation profiling are far less accurate, sensitive, and efficient than popularly believed, and as a result the role of epigenetic factors in human biology remains poorly understood.

Most methylation analysis depends on bisulfite conversion (Clark 1994 and Lister 2009), which was introduced in 1993 and has been only slightly improved since then. In this method DNA is incubated at elevated temperature in strong alkali in the presence of sodium bisulfite, which attacks the 5-6 double bond in cytosine; this attack is blocked by methylation (or hydroxymethylation) at the 5 position. Bisulfite attack leads to oxidative deamination at the 4 position to convert cytosine directly to uracil; after PCR amplification, cytosines that were unmethylated in the starting DNA are sequenced as thymines. Bisulfite sequencing has several shortcomings that are usually ignored for the sake of convenience. First, alkali- and bisulfite-mediated DNA degradation is so severe that bisulfite conversion only approaches completion when >97% of the DNA is cleaved into fragments of <300 bp (Warnecke 2002). This means that bisulfite sequencing requires relatively large amounts of DNA and suited only to short read sequencing. Second, bisulfite attack at unmethylated cytosines leads to a higher incidence of strand breakage at these sequences, which strongly enriches for methylated sequences; the bias can exceed 10-fold (Grunau 2001). Third, there is an enormous loss of sequence complexity after bisulfite conversion because >95% of all cytosines are converted to thymines; it cannot be known whether a T in a given sequence read was a C or a T in the starting material. As a result, many C-rich single-copy sequences map to multiple locations in the genome after bisulfite conversion. Fourth, CpG dinucleotides in some sequence contexts are inherently resistant to bisulfite attack (Harrison 1998). Fifth, existing methods cannot cover the entire genome.

Kriukiene et al., 2013 is a published case in which DNA methyltransferases has been used in methylation detection. However, this published method can only identify DNA fragments that contain at least one unmethylated CpG dinucleotide and can contain any number of methylated sites. The method of Kriukiene cannot achieve single nucleotide resolution, and is incompatible with long read nanopore sequencing. In comparison, the method of the invention of this application is highly innovative in that it is the first method that can map all modified cytosines in the genome at single base resolution by novel technology that is suited to all extant nanopore sequencing platforms.

It has not been previously possible to obtain whole genome patterns of modified cytosines at single nucleotide resolution with acceptable levels of accuracy, sensitivity, and economy. There is a pressing need for a method that can detect all modified bases in the human genome in a manner that is faster, cheaper, and more accurate and sensitive than existing methods. Provided herein is a flexible and radically new method that uses single molecule nanopore sequencing to identify all modified cytosines in the genome with great increases in accuracy, economy, sensitivity, and throughput as compared to extant methods.

### SUMMARY OF THE INVENTION

The subject invention provides a method of determining whether a cytosine at a predefined position within a single strand of a double-stranded DNA of known sequence is hydroxymethylated comprising:
a) contacting the double-stranded DNA with a glucosyltransferase and a uridine diphosphate glucose (UDP-glucose) so as to replace the hydrogen of hydroxymethylated cytosine with the glucose if the cytosine is hydroxymethylated; and
b) determining whether the cytosine contains the glucose;
   wherein if the cytosine contains the glucose the cytosine is hydroxymethylated cytosine.

The invention also provides a method of determining whether a cytosine at a predefined position within a single strand of a double-stranded DNA of known sequence is unmethylated comprising:
a) treating the double-stranded DNA with an oxidizing agent so as to convert methylated cytosine into hydroxymethylated cytosine if cytosine is methylated;
b) contacting the treated double-stranded DNA from step a) with a glucosyltransferase and a uridine diphosphate glucose (UDP-glucose) so as to replace the hydrogen of the hydroxymethylated cytosine with the glucose if the cytosine is hydroxylated; and
c) determining whether the cytosine contains the glucose;
   wherein if the cytosine does not contain glucose the cytosine is unmethylated.

The invention further provides a method of determining whether a cytosine at a predefined position within a single strand of a double-stranded DNA of known sequence is methylated but not hydroxymethylated comprising:
a) first determining whether the cytosine is hydroxymethylated according to the methods disclosed herein; and
b) separately determining whether the cytosine is unmethylated according to the methods disclosed herein;
   wherein if the cytosine is neither hydroxymethylated nor unmethylated, it is methylated.

The invention also provides a method of determining whether a cytosine present at a predefined position within a single strand of a double-stranded DNA of known sequence, and within a CpG site, is unmethylated comprising:
a) treating the double stranded DNA with a methyltransferase and an S-adenosylmethionine analog having the structure:

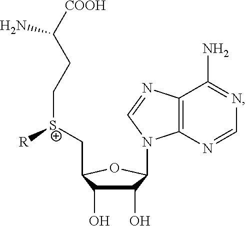

so as to replace the hydrogen attached to the 5 position of the cytosine with R if the cytosine is unmethylated and within a CpG site; and
b) determining whether the cytosine contains R;
   wherein if the cytosine contains R the cytosine is a unmethylated cytosine within a CpG site,
   wherein R is: an octadiynyl moiety,

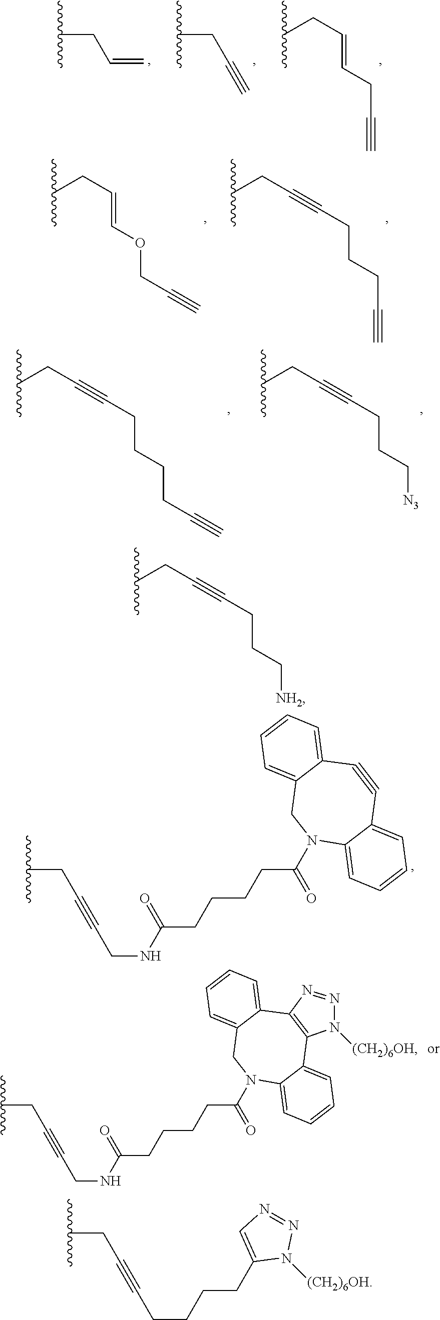

### BRIEF DESCRIPTION OF THE FIGURES

FIG. 1. Comprehensive analysis of cytosine modification. A: When only CpG methylation data is required, unmethylated CpG dinucleotides are labeled with a tag that gives a distinct signal during single molecule sequencing (SMS). B: To map all hydroxymethyl-Cs, a labeled sugar is transferred to the hydroxyl group with T4 βGT; C: To map all methylated cytosines, the 5-methyl group is oxidized with the catalytic domain of TET1 with simultaneous labeled sugar modification using T4 βGT in a single-tube reaction. Bases labeled in B are subtracted from those labeled in C to obtain a map of all CpG and CpN methylation.

FIG. 2. Principle of nanopore SBS. a: Nanopore-polymerase sequencing engine. A single DNA polymerase molecule is covalently attached to an α-hemolysin nanopore heptamer. Primer and template DNA (shown as a double-hairpin conformation) bind, along with tagged nucleotide, forming a complex with the polymerase. b: SBS schematic showing the sequential capture and detection of tagged nucleotides by the nanopore as they are being incorporated into the growing DNA strand in the polymerase reaction.

FIG. 3. Sequencing on nanopore array chips. Sequencing reactions were performed with inserted α-hemolysin pores conjugated to a single Phi29 DNA polymerase molecule, synthetic template, and the 4 tagged nucleotides. A: 4 bases are clearly distinguished. B: A 12-base homopolymer sequence is resolved. Events with dwell times shorter than those of actual incorporation events are recognized by the sequencing software and are not called. C: Newly incorporated nucleotides can be distinguished both by the electrical resistance provided by the tag and by the time required for incorporation of the nucleotide. The R indicates the label that is designed to delay incorporation of the complementary base.

FIG. 4: Effect of cytosine substitutions on polymerase extension rates. A: Template bearing a 5′ Cy3 dye contains either 6 CpG's, 6 5-methyl (Me)-CpG's or 6 5-octadiyne (Oct)-CpG's that the polymerase traverses during primer extension. Extension of a primer displaces a strand with a quencher at its 3′ end. Quencher strand displacement results in enhanced fluorescence. B: After pre-incubation in the presence of dNTPs and Bst 2.0 polymerase, MgCl₂ is added to start the reaction and fluorescence is recorded at the emission maximum (564 nm) with 548 nm excitation. Polymerase reaction rates reflected by t_{1/2} are in the following order: 92 s (CpG)<110 s (Me-CpG)<138 s (Oct-CpG). C: The incorporation is slowed due to crowding of the active site by the 5′ substitutions on C in CpG's.

FIG. 5: Label transfer by optimized mutants of M.SssI. A. View of the active site pocket of M.SssI modeled on the DNA-M.HhaI co-crystal (PDB 5MHT; (Klimasauskas 1994)) rendered in PyMol. The active site pockets of all DNA (cytosine-5) methyltransferases are highly conserved in sequence and structure (Goll 2005), and both the indicated Q and N are conserved in M.HhaI and M.SssI. A large pore or channel that connects solvent to the sulfur of AdoMet/AdoHcy is clearly visible; the pore is further enlarged by the Q142S and N370S substitutions. B. AdoMet analogs with propene and propyne substituents replacing the methyl group were synthesized as in FIG. 9. C. The SS and QS mutants can transfer labels from AdoMet derivatives to DNA, as shown by blockage of methylation-sensitive restriction endonucleases. Note that wild type M.SssI is inactive with these analogs. The SS and QS mutants show quantitative conversion. Only the (R) stereoisomers of the analogs were active. Analog 7 is as active as analog 1 in this assay (data not shown). D. Bisulfite sequencing shows quantitative conversion after transfer of analog 1 by SS mutant. In addition to the SS and QS mutants shown, AA, AS, QA, and AN mutants have been produced in the expectation that some AdoMet analogs will be more efficient substrates for specific M.SssI mutants.

FIG. 6: General scheme for transfer of bulky groups from AdoMet analogues to the C-5 position of CpG cytosines. Examples of side groups to replace the methyl group on S-adenosyl methionine are shown in FIG. 8.

FIG. 7: The overall scheme for methylation analysis by modification and single-molecule sequencing with the octadiyne R group as an example. In this case, which is a variant of that shown in FIG. 6, after transfer of the R group by the methyltransferase to the C-5 position of a CpG cytosine, click chemistry based capture (for example, with streptavidin beads, shown here as spheres) is used to decrease overall complexity of the molecules, i.e., nearly all will have originally been unmethylated cytosines. This will greatly reduce the amount of required sequencing. Thus, while the capture step is optional, it is highly recommended.

FIG. 8: Examples of side groups to replace the methyl group on S-adenosyl methionine are shown in this figure; representative synthetic schemes are described in FIG. 9.

FIG. 9: Example syntheses of R-group derivatized AdoMet analogues. AdoMet derivatives are generated by using chemistry similar to that described in Lukinavicius 2013.

FIG. 10: Examples of groups (ending in N₃ or alkyne) that can be attached to the C6 position on the sugar of UDP-glucose. After these molecules are transferred to 5-hydroxymethylcytosines by β-glucosyltransferase, click chemistry can be used to attach additional bulky groups with dibenzylcyclooctyne or N₃ respectively as described in Song 2012.

FIG. 11: Kinetic assay with 19.2 U of Bst 2.0. The fastest reaction took place with unmodified CpG's and the slowest reaction with six 5-Octadiynyl-CpGs. There was little difference in the reaction rates for extension reactions with three Me-CpG's, six Me-CpG's, three Prop-CpG's and six Prop-CpG's.

FIG. 12: Kinetic assay with 40 U of Bst 2.0. The fastest reaction took place with unmodified CpG's and the slowest reaction with six 5-Octadiynyl-CpGs. There was little difference in the reaction rates for extension reactions with three Me-CpG's, six Me-CpG's, three Prop-CpG's and six Prop-CpG's.

FIG. 13: Purification of M.SssI mutant SS using a His-Tag column. The conditions are optimized with additional purification steps, but this level of purification is sufficient for obtaining good transfer of AdoMet and AdoMet analogues to a human DNA PCR product as shown in FIG. 14.

FIG. 14: Transfer of groups from AdoMet and Prop-AdoMet to CpG Cytosines in double stranded DNA. After transfer from the AdoMet substrate to the DNA (cytosines in CpG sites), treatment of the DNA (containing a single CCGG site) with HpaII is carried out. HpaII will cleave only sites with unmodified cytosines. Lane 1: Untreated DNA. Lane 2: DNA+HpaII. Lane 3: DNA+wt M.SssI+HpaII, without AdoMet. Lane 4: DNA+AdoMet+wt M.SssI+HpaII. Lane 5: DNA+AdoMet+M.SssI mutant SS+HpaII. Lane 6: DNA+Prop-AdoMet+M.SssI mutant SS+HpaII. Near complete protection is observed in lanes 4, 5 and 6. Thus the wild-type enzyme can effectively transfer the only methyl groups to CpG cytosines, while the mutant enzyme can transfer either methyl or propargyl groups to CpG cytosines.

FIG. 15: Transfer of methyl groups from AdoMet to CpG Cytosines in E. coli DNA. An initial treatment of the E. coli DNA was carried out with BamHI to reduce the overall size. Then the DNA was incubated with AdoMet and either wild-type M.SssI (lane 4) or the SS mutant (lane 5) before treatment with HpaII. For comparison, lanes 3 and 6 show BamHI+HpaII treated DNA (without M. SssI treatment).

### DETAILED DESCRIPTION OF THE INVENTION

### Terms

As used herein, and unless stated otherwise, each of the following terms shall have the definition set forth below.

A—Adenine;

C—Cytosine;

DNA—Deoxyribonucleic acid;

G—Guanine;

RNA—Ribonucleic acid;

T—Thymine; and

U—Uracil.

“Nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Type” of nucleotide refers to A, G, C, T or U. “Type” of base refers to adenine, guanine, cytosine, uracil or thymine.

“Mutant” DNA methyltransferases refer to modified DNA methyltransferases including but not limited to modified M.SssI, M.HhaI and M.CviJI.

“Mass tag” shall mean a molecular entity of a predetermined size which is capable of being attached by a cleavable bond to another entity.

“Hybridize” shall mean the annealing of one single-stranded nucleic acid to another nucleic acid based on sequence complementarity. The propensity for hybridization between nucleic acids depends on the temperature and ionic strength of their milieu, the length of the nucleic acids and the degree of complementarity. The effect of these parameters on hybridization is well known in the art (see Sambrook J, Fritsch E F, Maniatis T. 1989. Molecular cloning: a laboratory manual. Cold Spring Harbor Laboratory Press, New York.)

As used herein, and unless otherwise stated, a “unmethylated cytosine” or a “cytosine that is unmethylated” or a “cytosine that is not methylated” refers to 4-aminopyrimidin-2(1H)-one.

As used herein, and unless otherwise stated, a “methylated cytosine that is not a hydroxymethylated cytosine” or a “cytosine that is methylated but not hydroxymethylated” refers to 5-methylcytosine (IUPAC name: 4-amino-5-methyl-3H-pyrimidin-2-one).

As used herein, a “hydroxymethylated cytosine” or a “cytosine that is hydroxymethylated” refers to 5-hydroxymethylcytosine (UPAC name: 6-amino-5-(hydroxymethyl)-1H-pyrimidin-2-one).

As used herein, and unless otherwise stated, a “methylated cytosine” or a “cytosine that is methylated” refers to either (a) 5-methylcytosine or (b) 5-hydroxymethylcytosine.

The subject invention provides a method of determining whether a cytosine at a predefined position within a single strand of a double-stranded DNA of known sequence is hydroxymethylated comprising:
a) contacting the double-stranded DNA with a glucosyltransferase and a uridine diphosphate glucose (UDP-glucose) so as to replace the hydrogen of hydroxymethylated cytosine with the glucose if the cytosine is hydroxymethylated; and
b) determining whether the cytosine contains the glucose;
   wherein if the cytosine contains the glucose the cytosine is hydroxymethylated cytosine.

The invention also provides a method of determining whether a cytosine at a predefined position within a single strand of a double-stranded DNA of known sequence is unmethylated comprising:
a) treating the double-stranded DNA with an oxidizing agent so as to convert methylated cytosine into hydroxymethylated cytosine if cytosine is methylated;
b) contacting the treated double-stranded DNA from step a) with a glucosyltransferase and a uridine diphosphate glucose (UDP-glucose) so as to replace the hydrogen of the hydroxymethylated cytosine with the glucose if the cytosine is hydroxylated; and
c) determining whether the cytosine contains the glucose;
   wherein if the cytosine does not contain glucose the cytosine is unmethylated.

The invention further provides a method of determining whether a cytosine at a predefined position within a single strand of a double-stranded DNA of known sequence is methylated but not hydroxymethylated comprising:
a) first determining whether the cytosine is hydroxymethylated according to the methods disclosed herein; and
b) separately determining whether the cytosine is unmethylated according to the methods disclosed herein;
   wherein if the cytosine is neither hydroxymethylated nor unmethylated, it is methylated.

In some embodiments, the oxidizing agent is ten-eleven translocation methylcytosine dioxygenase 1. In further embodiments, steps a) and b) occur simultaneously.

In additional embodiments, the glucose is labeled with a detectable chemical group. In further embodiments, glucose is labeled at position 6 with the chemical group. The chemical group may be a chemical group selected from the group consisting of: azide, detectable alkynyl, an alkyne,

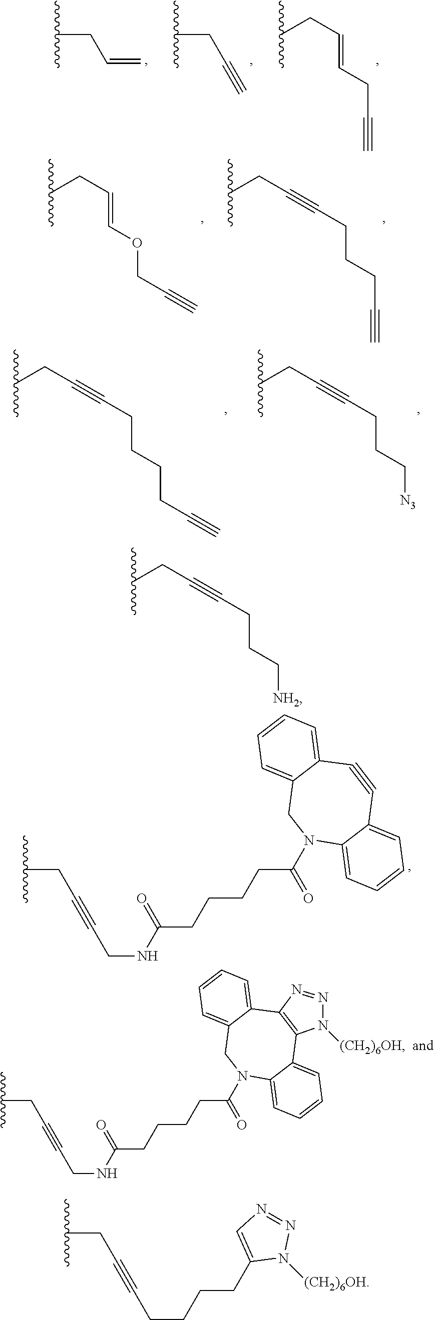

In some embodiments, the determining step comprises sequencing the single strand, which includes the hydroxymethylated cytosine with the glucose, with a single molecule sequencing technology. The single molecule sequence technology is able to differentiate between the hydroxymethylated cytosine with the glucose and other cytosines such as 5-Methylcytosine, 5-Hydroxymethylcytosine, and unmethylated cytosines.

The subject invention also provides a method of determining whether a cytosine present at a predefined position immediately adjacent to a guanine within a single strand of a double-stranded DNA sequence of known sequence is non-methylated comprising:
a) obtaining such a double-stranded DNA of known sequence comprising a cytosine at such predetermined position immediately adjacent to a guanine in such single strand;
b) producing a derivative of such double-stranded DNA by contacting the double-stranded DNA with a methyltransferase and an S-adenosylmethionine analog having the structure:

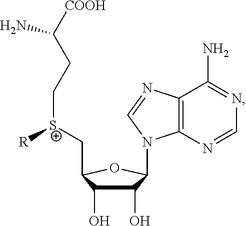

c) wherein R is a chemical group capable of being transferred from the S-adenosylmethionine analog by the methyltransferase to a 5 carbon of a non-methylated cytosine within the double-stranded DNA so as to covalently bond the chemical group to the 5 carbon of the non-methylated cytosine of the double-stranded DNA, thereby making a modified cytosine within the derivatized double stranded DNA,
d) wherein a single molecule sequencing technology is able to detect the difference between a methylated cytosine and the modified cytosine within the derivatized double stranded DNA, and
using the single molecule sequencing technology to determine whether a cytosine present at a predefined position immediately adjacent to a guanine within a single strand of a double-stranded DNA sequence of known sequence is non-methylated.

In one embodiment, the method further comprises a step of
i. separately obtaining a single strand of the derivative of the double-stranded DNA;
ii. sequencing the single strand so obtained in step i) with a single molecule sequencing technology; and
iii. comparing the sequence of the single strand determined in step ii) to the sequence of a corresponding strand of the double-stranded DNA of which a derivative has not been produced,
wherein the modification of the cytosine in the single strand of the derivative indicates that the cytosine at the predefined position in the single strand of the double-stranded DNA is non-methylated.

In some embodiments, the methyltransferase is a mutant M.SssI methyltransferase, a mutant CpG-specific methyltransferase or a C5-specific methyltransferase. The C5-specific methyltransferase may be is selected from the group consisting of M.HhaI, DNMT1, DNMT3A, DNMT3B, and biologically active analogs of the foregoing.

The invention also provides a method of determining whether a cytosine present at a predefined position within a single strand of a double-stranded DNA of known sequence, and within a CpG site, is unmethylated comprising:
a) treating the double stranded DNA with a methyltransferase and an S-adenosylmethionine analog having the structure:

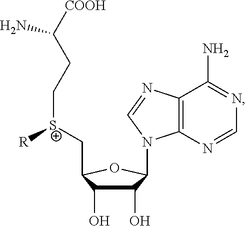

so as to replace the hydrogen attached to the 5 position of the cytosine with R if the cytosine is unmethylated and within a CpG site; and
b) determining whether the cytosine contains R;
   wherein if the cytosine contains R the cytosine is a unmethylated cytosine within a CpG site,
   wherein R is: an octadiynyl moiety,

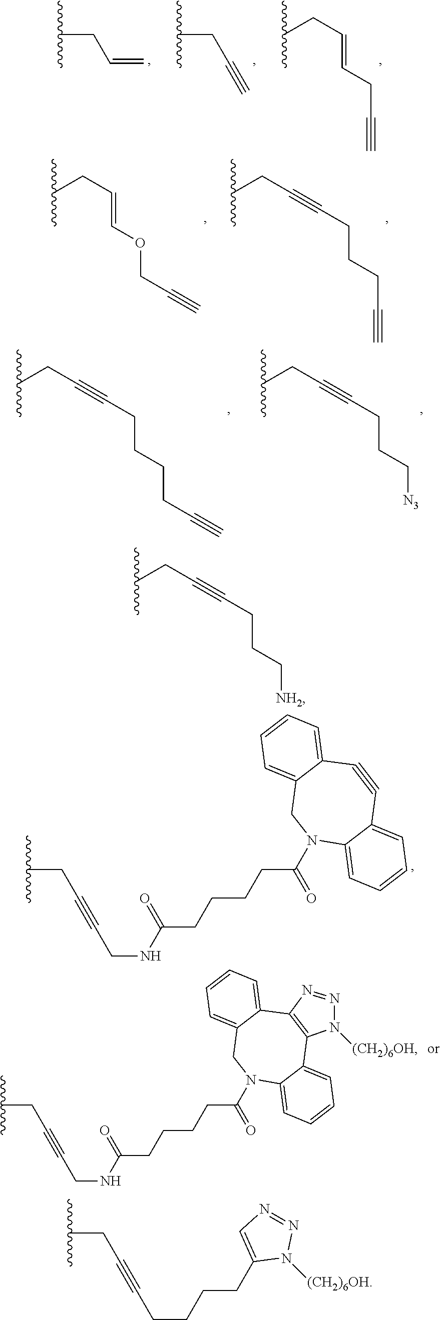

In embodiments, the method is performed without producing (i) a U analog by photo-conversion, (ii) a thymidine analog, or (iii) a neobase.

In additional embodiments, R is a propargyl group and the method further comprises adding an azido compound to the propargyl group by click chemistry

The invention also provides a method of determining whether a cytosine present at a predefined position within a single strand of a double-stranded DNA sequence of known sequence is hydroxymethylated comprising:
a) obtaining such a double-stranded DNA of known sequence comprising a cytosine at such predetermined position in such single strand;
b) producing a derivative of such double-stranded DNA by contacting the double-stranded DNA with a glucosyltransferase so as to covalently bond a sugar or a labeled sugar to the hydroxyl group of the 5 carbon of the hydroxymethylated cytosine of the double-stranded DNA, thereby making a modified hydroxymethylated cytosine within the derivatized double stranded DNA,
c) wherein a single molecule sequencing technology is able to detect the difference between a non-methylated or methylated cytosine and the modified hydroxymethylated cytosine within the derivatized double stranded DNA and using the single molecule sequencing technology to determine whether a cytosine present at a predefined position immediately within a single strand of a double-stranded DNA sequence of known sequence is hydroxymethylated.

In some embodiments, the method further comprises a step of
i. separately obtaining a single strand of the derivative of the double-stranded DNA;
ii. sequencing the single strand so obtained in step i) with a single molecule sequencing technology; and
iii. comparing the sequence of the single strand determined in step ii) to the sequence of a corresponding strand of the double-stranded DNA of which a derivative has not been produced,
wherein the modification of the cytosine in the single strand of the derivative indicates that the cytosine at the predefined position in the single strand of the double-stranded DNA is hydroxymethylated.

The invention further provides a method of determining whether a cytosine present at a predefined position anywhere within a single strand of a double-stranded DNA sequence of known sequence is methylated or hydroxymethylated comprising:
a) obtaining such a double-stranded DNA of known sequence comprising a cytosine at such predetermined position in such single strand;
b) producing a oxidized derivative of such double-stranded DNA by oxidizing a methylated cytosine to form a hydroxymethylated cytosine,
c) producing a second derivative of such double-stranded DNA by contacting the oxidized derivative with a glucosyltransferase so as to covalently bond the chemical group to the hydroxyl group of the 5 carbon of the hydroxymethylated cytosine of the oxidized derivative, thereby making the modified hydroxymethylated cytosine within the second derivatized double stranded DNA,
d) wherein a single molecule sequencing technology is able to detect the difference between a non-methylated cytosine and the modified hydroxymethylated cytosine within the second derivatized double stranded DNA
using the single molecule sequencing technology to determine whether a cytosine present at a predefined position anywhere within a single strand of a double-stranded DNA sequence of known sequence is methylated or hydroxymethylated.

In one embodiment, the method further comprises steps of
i. separately obtaining a single strand of the second derivative of the double-stranded DNA;
ii. sequencing the single strand so obtained in step i) with a single molecule sequencing technology; and
iii. comparing the sequence of the single strand determined in step ii) to the sequence of a corresponding strand of the double-stranded DNA of which a derivative has not been produced,
wherein the modification of the cytosine in the single strand of the second derivative indicates that the cytosine at the predefined position in the single strand of the double-stranded DNA is methylated or hydroxymethylated.

In some embodiments, the step of oxidizing a methylated cytosine to form a hydroxymethylated cytosine comprises contacting the double-stranded DNA with the catalytic domain of TET1. Steps b) and c) may occur simultaneously. In some embodiments, the method can differentiate between a hydroxymethylated cytosine and an unmethylated cytosine.

In an embodiment, the glucosyltransferase is T4-glucosyltransferase.

The invention also provides a method of determining whether a cytosine present at a predefined position anywhere within a single strand of a double-stranded DNA sequence of known sequence is methylated comprising:
(a) determining whether the cytosine is methylated or hydroxymethylated,
(b) determining whether the cytosine is hydroxymethylated, thereby determining whether the cytosine is methylated.

In some embodiments, the method can differentiate between a methylated non-CpG cytosine, and an unmethylated cytosine.

In one embodiment, the single molecule sequencing technology is a single molecule nanopore sequencing technology. In another embodiment, the single molecule sequencing technology is PacBio® SMRT sequencing, Oxford Nanopore, or NanoSBS.

In certain embodiments, the single molecule sequencing technology is a sequencing platform which identifies nucleobases by polymerase kinetics, wherein the presence of a bulky group in the template strand reduces the activity of the DNA polymerase, resulting in longer inter-event duration in the region of the modification. NanoSBS™ is such a sequencing platform.

In other embodiments, the single molecule sequencing technology is a sequencing platform which identifies nucleobases by measuring current blockade signals as single-stranded DNA is translocated through a nanopore. Oxford Nanopore MinION® sequencing platform (often referred to as simply Oxford Nanopore) is such a sequencing platform.

In yet other embodiments, the single molecule sequencing technology is a sequencing platform which identifies nucleobases by the presence of base-specific fluorescent labels attached to terminal phosphates. PacBio® SMRT sequencing (often referred to as SMRT sequencing) is such a sequencing platform.

R may be a label, a bulky substituent, a charged substituent, an octadiynyl moiety, or a labeled sugar. In some embodiments, R is:

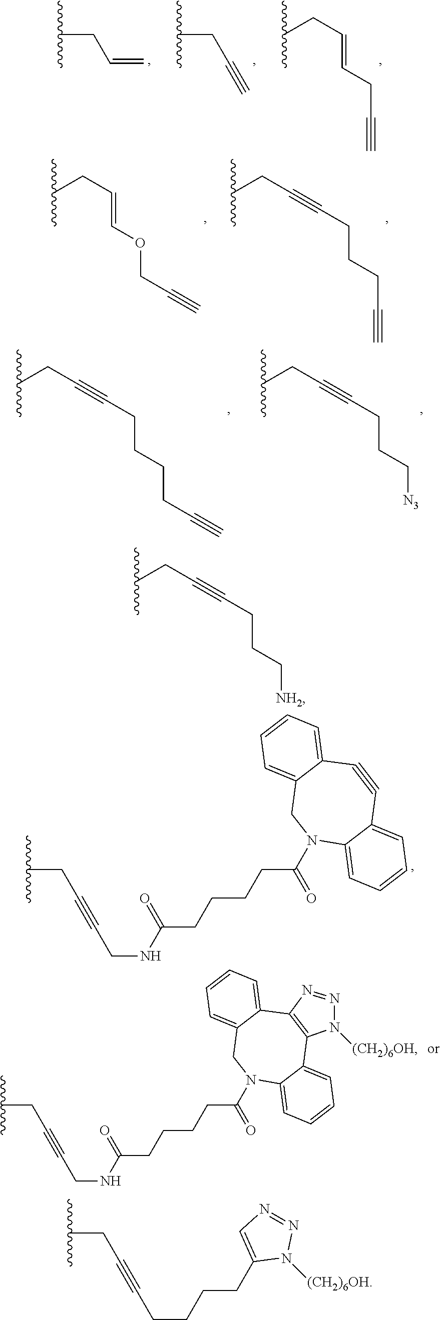

In certain embodiments, R is a propargyl group, i.e.

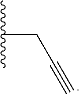 [SMILES: *CC#C]

In other embodiments, the method further comprises adding an azido group to the propargyl group by click chemistry. In some embodiments, the azido group is covalently linked to the alkyne of the propargyl group. In some embodiments, the addition of the azido group also improves the signal-to-noise ratio in the single molecule sequencing technology.

The invention further provides a compound having the following structure:

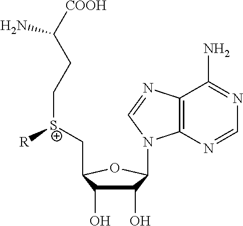

wherein R is

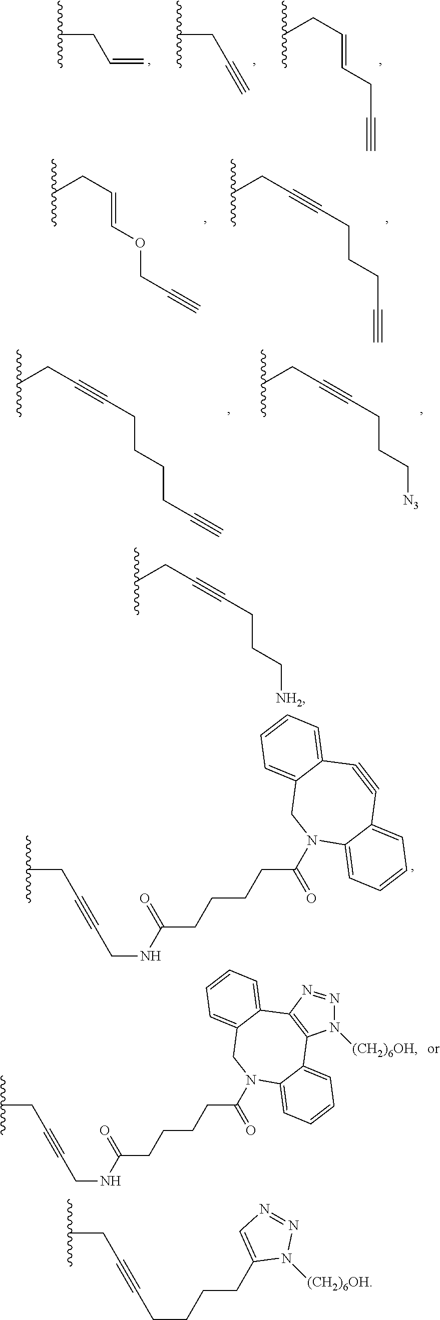

The invention also includes a composition comprising the compound.

The invention further provides a process of producing a derivative of a double-stranded DNA comprising contacting the double-stranded DNA with a methyltransferase and an S-adenosylmethionine analog having the structure:

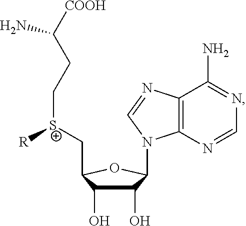

wherein R is a chemical group capable of being transferred from the S-adenosylmethionine analog by the methyltransferase to a 5 carbon of a non-methylated cytosine within the double-stranded DNA under conditions such that the chemical group covalently bonds to the 5-carbon of the non-methylated cytosine of the double-stranded DNA and thereby produces the derivative of the double-stranded DNA, wherein R has the structure:

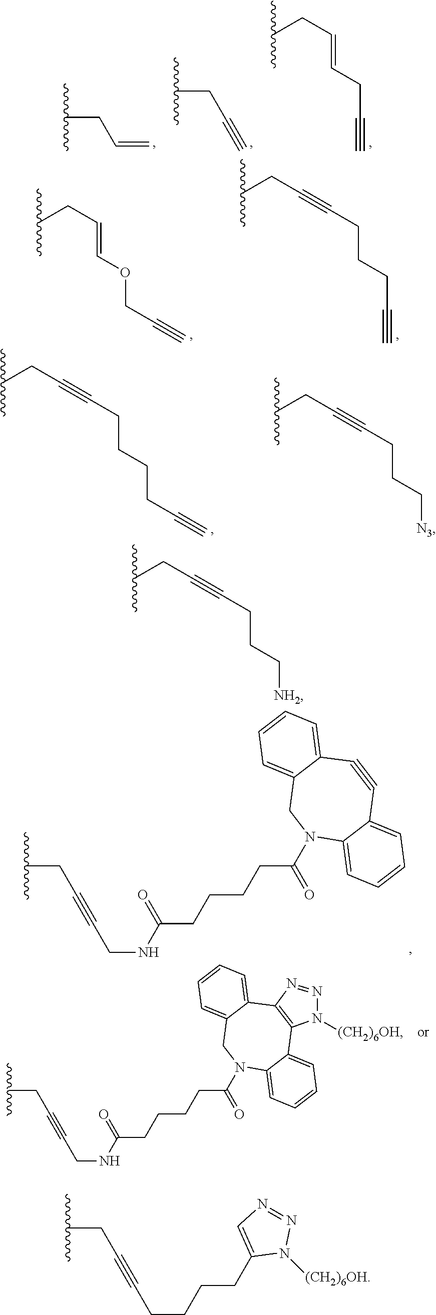

The methyltransferase may be a mutant M.SssI methyltransferase, a mutant CpG-specific methyltransferase, a C5-specific methyltransferase. The C5-specific methyltransferase may be selected from the group consisting of M.HhaI, DNMT1, DNMT3A, DNMT3B, and biologically active analogs of the foregoing.

In one embodiment, the chemical group capable of being transferred from the S-adenosylmethionine analog by the methyltransferase to a 5 carbon of a non-methylated cytosine within the double-stranded DNA permits a single molecule sequencing technology to determine the difference between a methylated cytosine and the cytosine covalently bonded to the chemical group.

The invention further provides a process of producing a derivative of a double-stranded DNA comprising contacting a double-stranded DNA, or a derivative thereof, with a glucosyltransferase and a uridine diphosphate glucose so as to replace the hydrogen of a hydroxymethylated cytosine with the glucose, wherein the glucose is labeled with a detectable chemical group selected from the group consisting of: an alkyne, azide, detectable alkynyl,

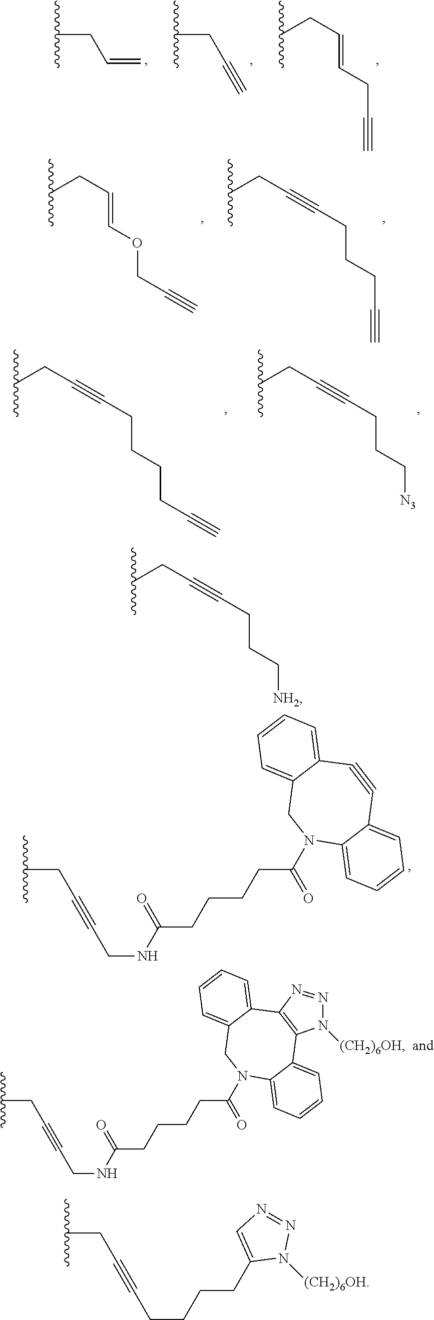

The invention further provides a process of producing a derivative of a double-stranded DNA comprising contacting a double-stranded DNA, or a derivative thereof, with a glucosyltransferase

In one embodiment, the glucosyltransferase is T4 β-glucosyltransferase.

In another embodiment, the glucose capable of being transferred permits a single molecule sequencing technology to determine the difference between an unmethylated cytosine and the hydroxymethylated cytosine covalently bound to the chemical group.

The present invention also provides a method for determining whether a cytosine at a predefined position within a single strand of a double-stranded DNA sequence of known sequence is non-methylated, methylated but not hydroxymethylated, or hydroxymethylated comprising
b) determining whether the cytosine is hydroxymethylated according to the methods disclosed herein; and
a) separately determining whether the cytosine is unmethylated according to the methods disclosed herein;
c) separately determining whether the cytosine is methylated but not hydroxymethylated according to the methods disclosed herein;
thereby determining whether the cytosine is either non-methylated, methylated or hydroxymethylated.

This invention provides methods for methylation profiling. Methods for methylation profiling are disclosed in U.S. Patent Application Publication No. US 2011-0177508 A1, which is hereby incorporated by reference.

This invention provides the use of DNA methyltransferases. Examples of DNA methyltransferases include but are not limited to M.SssI, M.HhaI and M.CviJI as well as modified M.SssI, M.HhaI and M.CviJI. These enzymes are modified mainly to have reduced specificity such that R groups on AdoMet analogs can be more efficiently transferred to unmethylated C residues, including in the context of a CpG site in DNA. Examples of such modified M.SssI and M.HhaI genes have been described in the literature (Lukinavicius et al 2012) Engineering the DNA cytosine-5 methyltransferase reaction for sequence-specific labeling of DNA. Nucleic Acids Res 40:11594-11602; Kriukene et al (2013) DNA unmethylome profiling by covalent capture of CpG sites. Nature Commun 4:doi:10.1038/ncomms3190).

Detectable tags and methods of affixing nucleic acids to surfaces which can be used in embodiments of the methods described herein are disclosed in U.S. Pat. Nos. 6,627,748, 6,664,079 and 7,074,597 which are hereby incorporated by reference.

Methods for production of cleavably capped and/or cleavably linked nucleotide analogues are disclosed in U.S. Pat. No. 6,664,079, which is hereby incorporated by reference.

DNA Methylation is described in U.S. Patent Application Publication No. 2003-0232371 A1 which is hereby incorporated by reference in its entirety.

Other Methods for determining the methylation status are disclosed in U.S. Patent Application Publication No. 2016-0355542-A1, which is hereby incorporated by reference in its entirety.

All combinations and subcombinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

Overview

DNA methylation expands the information content and modifies the function of the human genome. Genomic methylation patterns are abnormal in a number of human diseases, with the most extreme abnormalities found in cancer genomes. There is currently no efficient method for accurate genomic methylation profiling when only small amounts of DNA are available, and the standard method (bisulfite sequencing) badly overestimates methylation levels and has a high false positive rate. Our novel approach combines chemistry, enzymology and single molecule real-time sequencing platforms (i.e. Pacific Biosciences (PacBio®) SMRT sequencing, nanopore-based sequencing-by-synthesis NanoSBS™) to identify genome-wide CpG and non-CpG methylation and hydroxymethylation patterns. NanoSBS utilizes a different polymer tag on the terminal phosphate of each of the 4 bases in DNA. During nucleotide incorporation in the polymerase reaction, the tags differentially block current through a protein nanopore. The current blockade depth identifies the base, and the enzymatic addition of a larger chemical moiety to the 5 position of the specific cytosines will identify the modification status of that cytosine. This novel technology identifies all modified cytosines with much higher sensitivity, accuracy, efficiency, and economy when compared to extant methods. The presence of bulky groups can also serve to substantially amplify the signal due to unmethylated, methylated or hydroxymethylated cytosines in the Oxford Nanopore strand sequencing approach. In this example, as well as in Examples 2 and 3, a reference to a methylated cytosine generally refers to 5-methylcytosine. However, each reference to methylated cytosines should be viewed in the context of the surrounding text.

This example has four subsections, as follows:

Subsection 1: Model templates are synthesize bearing cytosines with labels at the C-5 position that produce time resolved signatures in single molecule sequencing (SMS) to identify modified cytosines in genomic DNA. Initial studies are performed with an octadiynyl moiety attached to the C-5 position of dC. Other bulky or charged substituents are also tested. Labels that give the most distinct and consistent time signatures during NanoSBS or SMRT sequencing are identified.

Subsection 2: M.SssI methyltransferase is optimized for transfer of bulky labels by site directed mutagenesis. AdoMet derivatives that deliver the labels optimized in subsection 1 are synthesized. Modifications in the binding pocket of methyltransferases have been shown to permit transfer of bulky moieties that replace the methyl group on synthetic analogs of S-adenosyl L-methionine (AdoMet). Mutant forms of the enzyme M.SssI (which methylates all CpG dinucleotides) that bear enlarged cofactor binding sites to obtain optimal rates of transfer of label from AdoMet analogs are screen. Mutant enzymes that mediate efficient transfer of an allyl, propyne and propene labels from AdoMet analogs have been obtained.

Subsection 3: Current blockade group transfer followed by NanoSBS on test DNAs with methylated and unmethylated CpGs to test the complete protocol is performed. The side groups that can be recognized and transferred from an AdoMet analog to cytosine by a mutant M.SssI and result in different time signatures for nucleotide incorporation during NanoSBS or SMRT sequencing compared to unmodified and methylated cytosines, are used in this analysis.

Subsection 4: NanoSBS approach is used for detection of 5-hydroxymethyl cytosines and all genomic methylated (CpG and non-CpG) cytosines. Though CpG methylation is by far the most common and most important epigenetic mark on DNA, hydroxymethylation of CpG cytosines and non-CpG methylation (CpN methylation) may also have biological functions. For hydroxymethyl cytosine detection, a labeled sugar is coupled onto the hydroxymethyl group using T4 β-glucosyltransferase. For non-CpG (in addition to CpG) methyl cytosine detection, the methyl group is oxidized to hydroxymethyl with the catalytic domain of TET1 dioxygenase.

These four subsections provide a method for the identification of all modified cytosines in genomic DNA that is highly superior to existing methods. Such an improved method will be essential to gain an understanding of the function of epigenetic factors in human health and disease.

This example provides the first system that allows identification of all modified cytosines by nanopore single molecule sequencing (SMS). SMS avoids amplification biases, can provide ultra-long (megabase) reads, and is much less expensive than Sanger or next-gen sequencing.

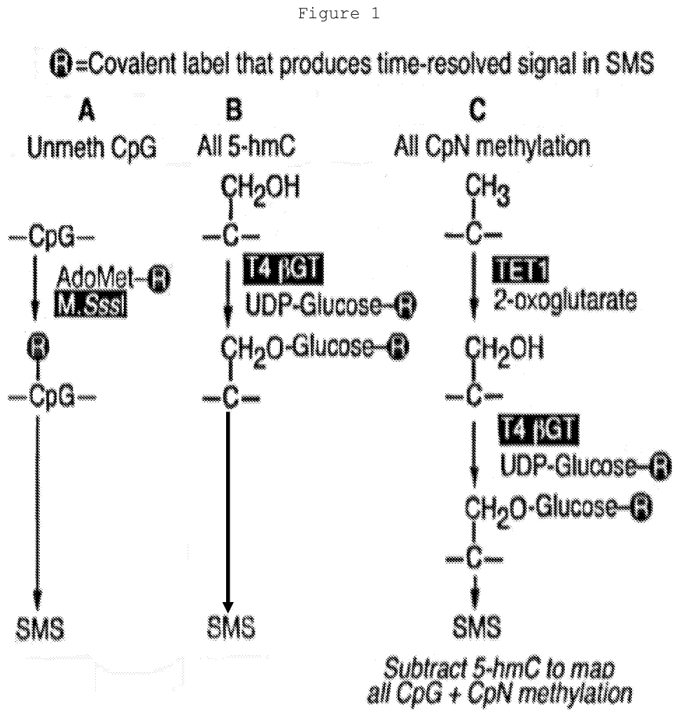

The approach is equally suited to several current SMS systems including the real-time single-molecule sequencing-by-synthesis strategy called NanoSBS (Kumar 2012, Fuller 2015, and Fuller 2016). In the case of unmethylated cytosines in CpG dinucleotides, the ability of mutant CpG-specific methyltransferases to transfer chemical labels from AdoMet analogs to the 5-position of cytosine is taken advantage of (Fuller 2015). For direct detection of hydroxymethylcytosine, a labeled sugar is attached to the hydroxymethyl position using T4 β-glucosyltransferase (βGT) (Flusberg 2010 and Li 2012). For genome wide methylcytosines (in both CpG and CpN contexts) a combined treatment with a TET1 catalytic domain dioxygenase to hydroxylate the methyl group, followed by sugar transfer by βGT (Nifker 2015 and Wu 2015) is used. The method is diagrammed in FIG. 1. Note that the approaches shown in A and in B and C are independent and alternative approaches. A will map all CpG methylation; B and C combined will map all CpG and CpN methylation and 5hmC in all sequence contexts.

A major advantage of the single molecule sequencing approach is the absence of amplification biases, which can be severe in PCR-dependent methods. In addition, enzymes rather than harsh chemicals are used to treat the DNA, all but eliminating DNA degradation-associated biases. Finally the technique is platform-agnostic with different single molecule sequencing systems; the method is used with NanoSBS technology and Pacific Biosciences' PacBio® SMRT sequencing. The NanoSBS approach is preferably used for the sequence readout in this study (Kumar 2012, Fuller 2015, and Fuller 2016).

This invention comprises 1) the first method that can provide accurate DNA modification profiling by nanopore sequencing, 2) the first method designed to minimize DNA damage which will greatly increase sensitivity, 3) the first method designed to be effective in all or nearly all single molecule sequencing platforms, 4) the first method that can identify all or nearly all modified cytosines in any sequence context, and 5) the first method that obviates amplification biases.

Approach

The predominant and most important cytosine methylation fraction in adult tissue occurs within a CpG context and is typically found within CpG islands in gene regulatory regions of the genome. But methylcytosines in CpN sequences and hydroxymethylated CpGs can reach 25% or more of the total modified cytosines in stem cells and in the adult central nervous system (Lister 2009, Kinde 2015, Kriaucionis 2009, and Tahiliani 2009). A sequencing method (NanoSBS) is used in which the bases of DNA are decoded in real time during the polymerase extension reaction by taking advantage of nanopore-discriminable polymer tags (Kumar 2012, Fuller 2015, and Fuller 2016). The enzymatically modified cytosines will retard the polymerase extension reaction, resulting in distinct time-resolved nanopore signatures for each modified base during NanoSBS and SMRT sequencing.

DNA (cytosine-5) methyltransferases transfer methyl groups from S-adenosyl L-methionine (AdoMet) to the 5 carbon of cytosine. Substitution of large amino acids with small amino acids in the active site pocket of the CpG-specific M.SssI allows transfer of larger S-substituted labels in AdoMet analogs. This finding is used to transfer bulky labels to unmethylated CpG cytosines, which will elicit altered polymerase reaction rates during NanoSBS.

It has been reported by PacBio® that methylcytosines and hydroxymethylcytosines in a template strand can slightly retard extension by DNA polymerase during SMRT sequencing, resulting in inter-event durations at or beyond the position of the altered base (Wallace 2010, Plongthongkum 2014, Schreiber 2013, Davis 2013, Clark 2012, Feng 2013, Schadt 2013, and Wu 2015). However, the signal provided by small methyl and hydroxymethyl moieties is weak and large false negative and false positive error rates are almost certain. It is notable that no published mammalian genomic methylation profiles have actually been obtained by current implementations of SMRT, and the indications are that signal-to-noise ratios will be too small. Molecular labels are developed that yield much larger signal differences that will provide accurate and sensitive identification of modified cytosines with PacBio® SMRT, Oxford Nanopore and NanoSBS sequencing platforms.

The overall approach is shown in FIG. 1, which emphasizes the placement of labels on the 5 carbon of cytosine by taking advantage of a CpG-specific methyltransferase to label unmodified CpG dinucleotides, β-glucosyltransferase to label hydroxymethylcytosine, and a combination of the catalytic domain of TET1 and T4 β-glucosyltransferase to label all CpG and CpN methylcytosines. There are several reports of the use of T4 β-glucosyltransferase (βGT) to transfer modified sugars to 5-hydroxymethyl cytosine (Li 2012 and Nifker 2015).

Preliminary Results:

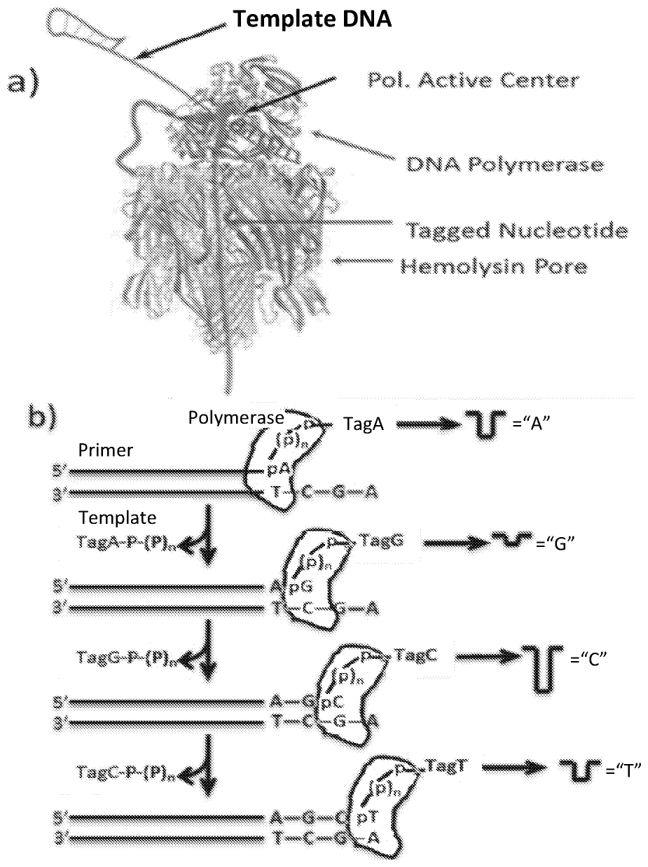

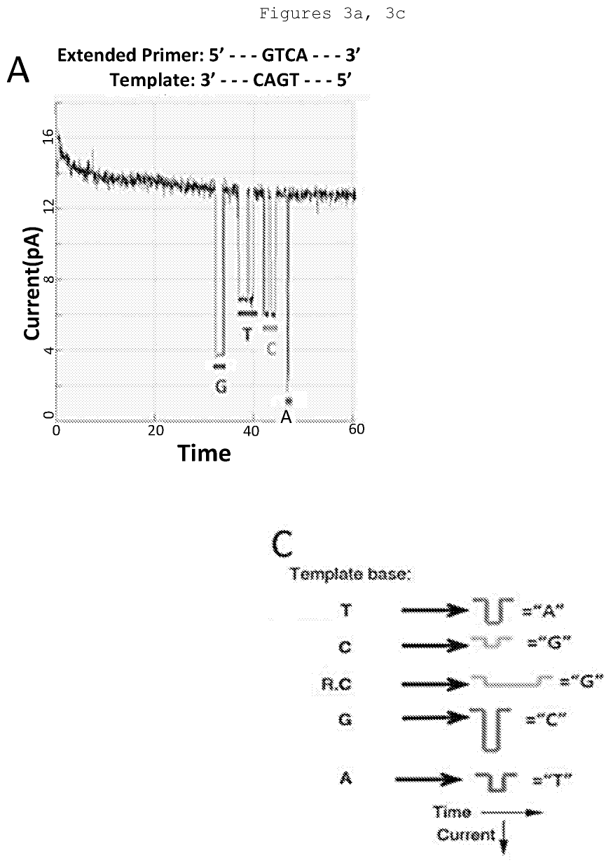

A sequencing method called nanopore sequencing-by-synthesis (NanoSBS) is depicted in FIG. 2 (Kumar 2012, Fuller 2015, and Fuller 2016). DNA polymerase is covalently attached to a protein nanopore (α-hemolysin) and incubated with template, primer and 4 tagged nucleotides. The nucleotides are hexaphosphates with polymer tags attached to the terminal phosphate. During the time a nucleotide is being incorporated, its tag is drawn by an applied voltage into the channel of the nanopore, where it impedes the current. Specific modifications of the polymer tags yield a different current blockade for each of the 4 nucleotides to allow sequence determination (FIG. 3).

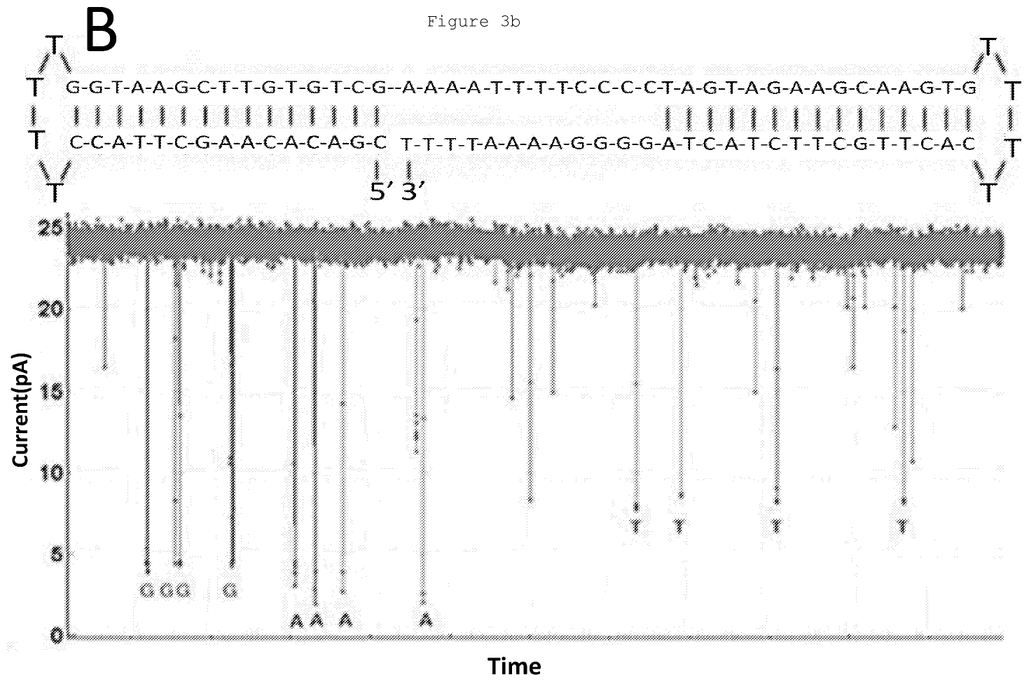

It has been demonstrated that placement of a methyl group or an octadiynyl group on CpG cytosines in synthetic template strands of DNA results in progressive slowing of polymerase in solution kinetic assays. A set of identical templates was created with 6 CpG's and a 5′-terminal fluorophore (Cy3), differing only in the absence or presence of one of the above groups on the 5-position of these 6 cytosines. A primer extension was used to displace a bound strand with a quencher at its 3′ end, where it is in proximity to the Cy3 when annealed to the template strand (FIG. 4), to demonstrate that the presence of 5-methyl cytosines and especially 5-octadiynyl cytosines has a significant slowing effect (on the order of tens of seconds) as measured by the t_{1/2} for loss of quencher and full development of fluorescence. These data indicate that the described labeling approach is effective.

Subsection 1

Templates bearing cytosines with modifications at the C-5 position that display characteristic time signatures in Nano-SBS relative to 5-MeC and unmodified C are synthesized. Unmethylated CpGs are distinguished from methylated CpGs. To do this, isolated DNA is incubated with AdoMet analogs bearing synthetic labels which can be transferred to the 5 carbon of cytosine.

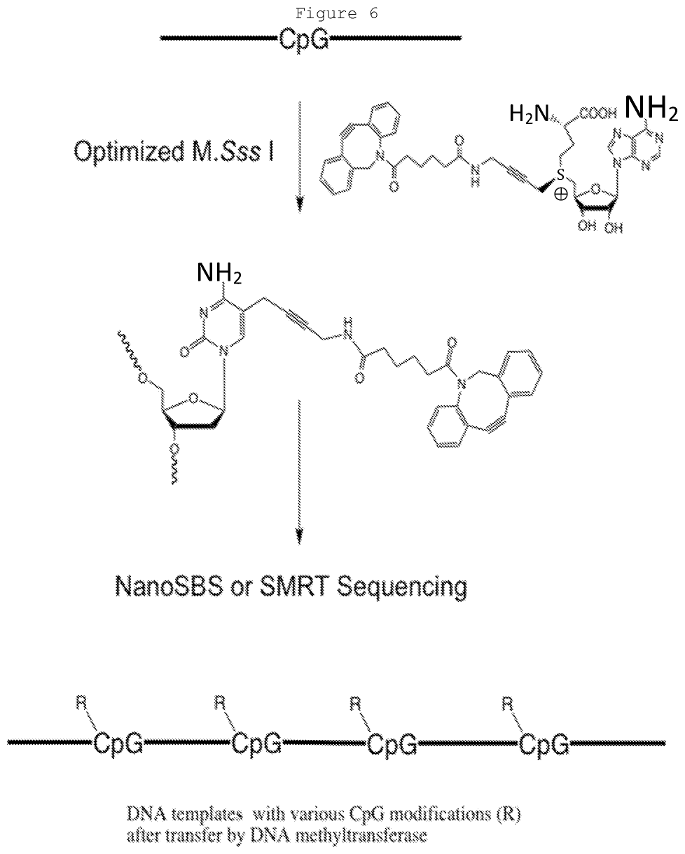

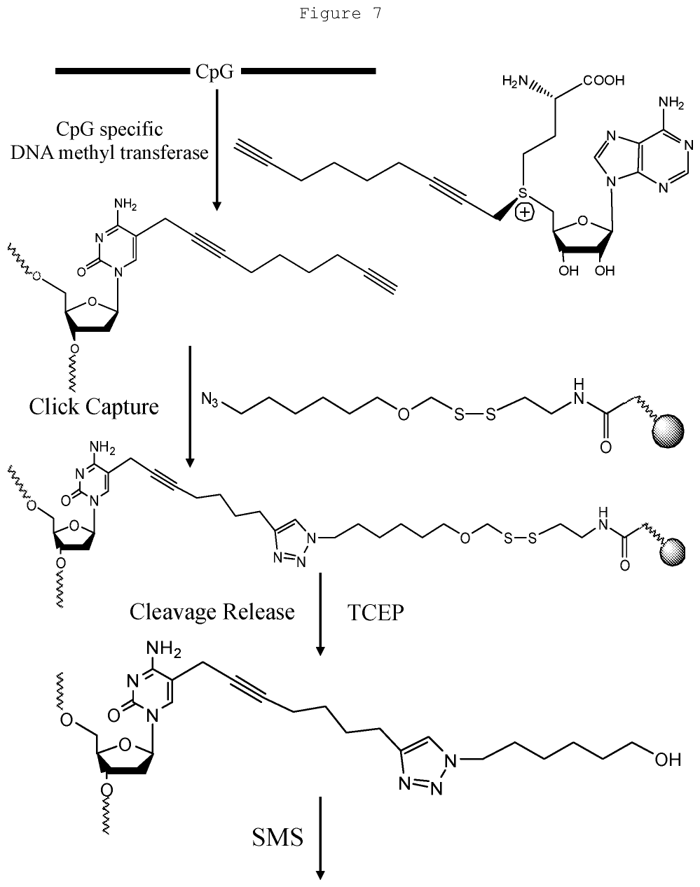

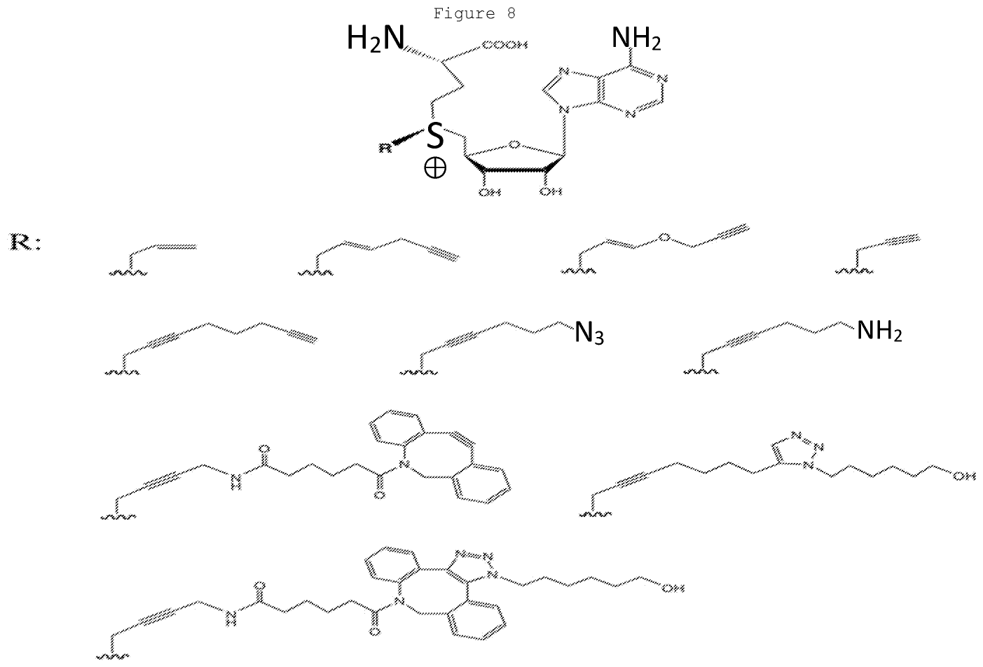

Synthetic compounds (cytosines bearing labels predicted to produce time-resolved signatures) are tested using solution-based polymerase reaction assays. Examples of potential groups based on the literature (Kriukiene 2013) are shown in FIG. 8; a typical scheme for their synthesis is shown in FIG. 9. Many other moieties can be easily synthesized and tested. A simple strand displacement assay involving fluorescence quenching (as in FIG. 4) and/or gel mobility shifts are used. Additionally, attachment of biotin for selection by streptavidin beads is used which permits capture and high throughput sequencing of just the CpG fraction of interest (see, for instance, FIG. 7). The biotin can be attached using a variety of chemical conjugation methods comprising azide-alkyne, tetrazine-cyclooctene, or azide-dibenzyl cyclooctyne click chemistry, amine-NHS ester, etc. Substitutions that slow polymerase reaction rates significantly below those found with unmodified cytosines, methylcytosines, and hydroxymethyl cytosines are identified. It is important to note that substituents at the C-5 position will not prevent normal base pairing. Following the solution assays, the best molecules are tested using the PacBio® SMRT system and the NanoSBS system.

Subsection 2

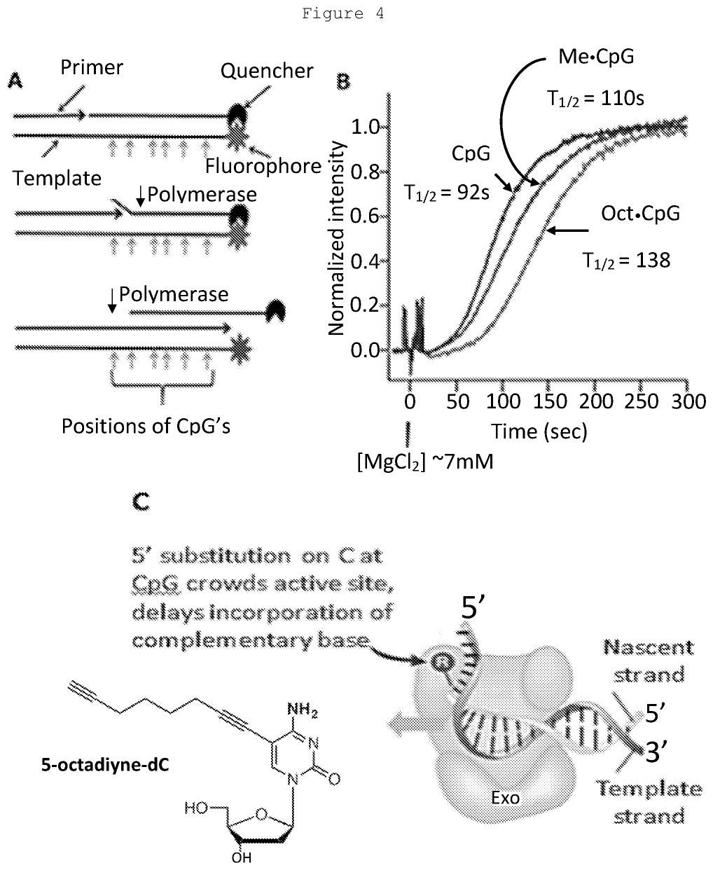

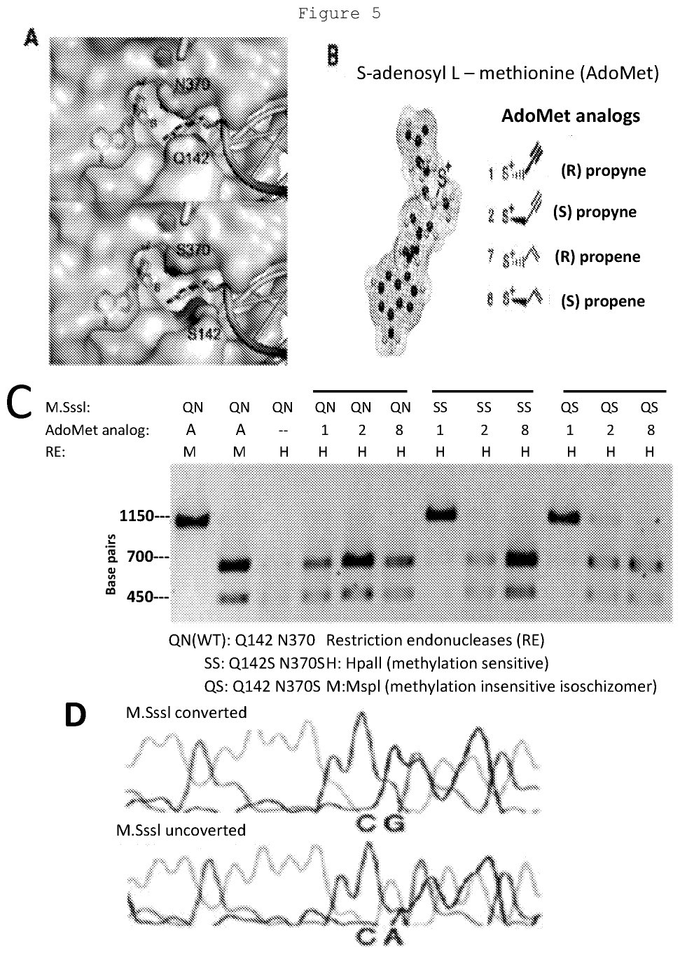

M.SssI methyltransferase is optimized for transfer of bulky labels by site directed mutagenesis of the active site pocket. A series of mutants of M.SssI, a bacterial methyltransferase that modifies all CpG sites (Renbaum 1990) have been constructed. An M.SssI expression construct was used. This bacterial plasmid construct contained the full open reading frame for M.SssI behind the Tac promoter (an inducible promoter that causes expression of S.SssI in E. coli upon exposure to isopropylthiogalactoside) as described in Clark 2012. As shown in FIG. 5, the mutant enzymes are much more efficient than the native enzyme in the transfer of bulky R groups, as had been reported in another study (Kriukiene 2013). FIG. 5A shows that a large pore connects the AdoMet binding site to the surrounding solvent, and after enlargement of the active site pocket bulky sulfonium-linked R groups will extend out through this pore without interfering with AdoMet analog binding. FIG. 5C shows that the (R) stereoisomers of propene and propyne R groups (synthetic schemes for synthesis of these AdoMet analogs are shown in FIG. 9) are transferred with very high efficiency by mutant M. SssI with an enlarged active site (lanes 7 and 10), while native M.SssI is almost completely inactive (lane 4). Efficient transfer of even larger R groups occurs, similar to what has been reported by others (Kriukiene 2013). Though the work described herein focuses on the bacterial enzyme M.SssI because it is well characterized and is specific for all CpG's, several other C5-specific methyltransferases exist including M.HhaI and the eukaryotic enzymes DNMT1, DNMT3A and DNMT3B. Though they all have somewhat different substrate specificities (M.HhaI methylates the first C in GCGC sequences, and the maintenance methylase DNMT1 preferentially methylates hemimethylated DNA), they can all be subjected to rational mutagenesis strategies that would make them more suitable for the purposes of the methods described herein. Other methyltransferases in bacteria produce N6-methyladenine or N4-methylcytosine, for which similar strategies can be developed. Straightforward assays are carried out to determine the ability of the mutant enzyme to transfer the label from the AdoMet analog to appropriate cytosines in double-stranded DNA. The appropriate label for cytosine that is identified in subsection 1 is placed on an AdoMet analog. The synthesis and use of such substituents are shown in FIGS. 6-9. This allows one to screen for the most efficient mutated M.SssI. First, the label transferred is tested for the acquisition of resistance to methylation-sensitive restriction endonucleases, as is shown in FIG. 5. To confirm that both strands have been converted at CpG dinucleotides, strand-specific bisulfite sequencing is appropriate as any covalent modification of the 5 position of cytosine prevents bisulfite attack; the drawbacks of bisulfite sequencing in whole-genome sequencing will not affect this assay. Results indicate that both strands are modified even when bulky labels are transferred (Kriukiene 2013).

Subsection 3

Enzyme-mediated label transfer is carried out followed by SMS on test DNAs with methylated and unmethylated CpGs to optimize the protocol. Using the preferred chemical group as ascertained by its effect on polymerase reaction rate (subsection 1) and ability to be transferred to unmethylated CpG cytosines by mutant M.SssI (subsection 2), the complete system from group transfer to capture of modified DNA to NanoSBS or SMRT sequencing is demonstrated. The approach is shown in FIGS. 1 and 7.

DNA containing labeled CpG dinucleotides are subjected to SMRT and NanoSBS sequencing. The latter can be performed on nanopore array chips. These sensor arrays contain individually addressable membranes with arrays of single nanopores. The DNA templates are isolated and converted to circular molecules or dumbbell-shaped structures using adapters that will serve as priming sites for sequencing reactions. The four tagged nucleotides are added in appropriate buffer enabling polymerase activity and ion conductance determination in the presence of an applied voltage gradient. As a nucleotide complementary to the template strand is being incorporated into the growing DNA (primer) strand, its tag is drawn into the channel of the nanopore, reducing the current to an extent specific to that tag, before being removed upon formation of the phosphodiester bond. The time between each current blockade event is also part of the readout. Differences in inter-event duration (IED) is measured as the polymerase passes the modified cytosines and for ˜10 bases thereafter relative to the IEDs near the equivalent unmodified cytosine in an untreated sample. Initial experiments are carried out on plasmid DNA with predetermined patterns of methylated and hydroxymethylated cytosines.

The approach outlined here is not limited to a specific single molecule sequencing platform. In addition to the Genia® Nanopore SBS system, it is conducive to sequencing using the Pacific Biosciences SMRT system as well as Oxford Nanopore's strand sequencing platform. In FIG. 1, they are described using the M.SssI bulky group transfer to unmethylated cytosines in CpG context (left side of FIG. 1), but the strategies for labeling methylcytosines, hydroxymethylcytosines, and context-independent unmethylated cytosines by taking advantage of sugar transfer reactions (right side of FIG. 1 and subsection 4) are expected to work equally well. Indeed, PacBio® has already reported the use of T4 β-glucosyltransferase-abetted transfer of UDP-glucose with capturable groups to 5-hydroxymethyl cytosines for sequencing in their system (Song 2012).

For the PacBio® system, which measures the presence of base-specific fluorescent labels attached to terminal phosphates in zero mode waveguides, the approach is essentially identical and like with Nanopore SBS, is based on polymerase kinetics, whereby the presence of a bulky group in the template strand reduces the activity of the DNA polymerase, resulting in longer inter-event duration in the region of the modification. As with Nanopore SBS, circularization of templates (e.g., using the SMRT method) for the subsequent sequencing is preferred and amplification should be avoided.

For nanopore strand sequencing, use of bulky groups has a different purpose. In the Oxford Nanopore system, the four nucleotides are distinguished by their differential effects on ion conductance through the nanopore. The depths of the ion current blockades elicited by A, C, G and T are fairly similar. Moreover, 5-6 consecutive bases are read simultaneously, limiting the overall accuracy of this approach. Two directed studies have shown the ability to detect 5-MeC and 5-OHMeC in an MspA nanopore (Schreiber 2013 and Laszlo 2013). More recently, it has been reported that the Oxford Nanopore sequencing engine can be used to distinguish cytosines from 5-methylcytosines (Simpson 2017, Rand 2017, Stoiber 2016), and 5-hydroxymethylcytosines (Rand 2017) with accuracy rates higher than 90% in some cases using high stringency thresholds. However, if M.SssI is used to transfer bulky groups to the 5-position of unmethylated CpG cytosines as described herein, these modified cytosines should have a much different ionic blockade level than cytosines alone. A sequence comparison in the absence and presence of complete bulky group transfer should provide strong evidence for the positions of methylated and unmethylated cytosines in CpG's. This method may also be used to specifically attach bulky groups to 5-MeC and 5-OHMeC using the UDP glucosyl transfer reaction approach with initial Tet1 oxidase treatment in the case of 5-MeC. For the Oxford system, linear single stranded DNA will be used. Additionally, with the strand sequencing approach, there may be a second built-in check. Since strand sequencing uses polymerase or helicase ratcheting approaches to slow movement of the DNA through the channel, one might also consider the effect of bulky side groups on their rates, keeping in mind that the position where the nucleotides thread through the polymerase are a set distance from the position in the channel where the signatures are obtained.

The choice of DNA polymerase to use is mainly determined by the DNA sequencing method itself. Generally, for single molecule methods, a highly processive enzyme is desirable. However, in theory, any polymerase that would be slowed by the presence of bulky side groups in the DNA template would be amenable to this approach.

Subsection 4

The NanoSBS approach is used for detection of 5-hydroxymethyl cytosines and all genomic methylated (CpG and non-CpG) cytosines. As mentioned earlier, CpG methylation is the most salient epigenetic DNA modifications in mammals. However, 5-hydroxymethyl CpG cytosines and non-CpG methylcytosines occur at a fairly high frequency in some cell types. These can be directly addressed by taking advantage of two enzymes, T4 β-glucosyltransferase (βGT) and the catalytic domain of TET1 dioxygenase.

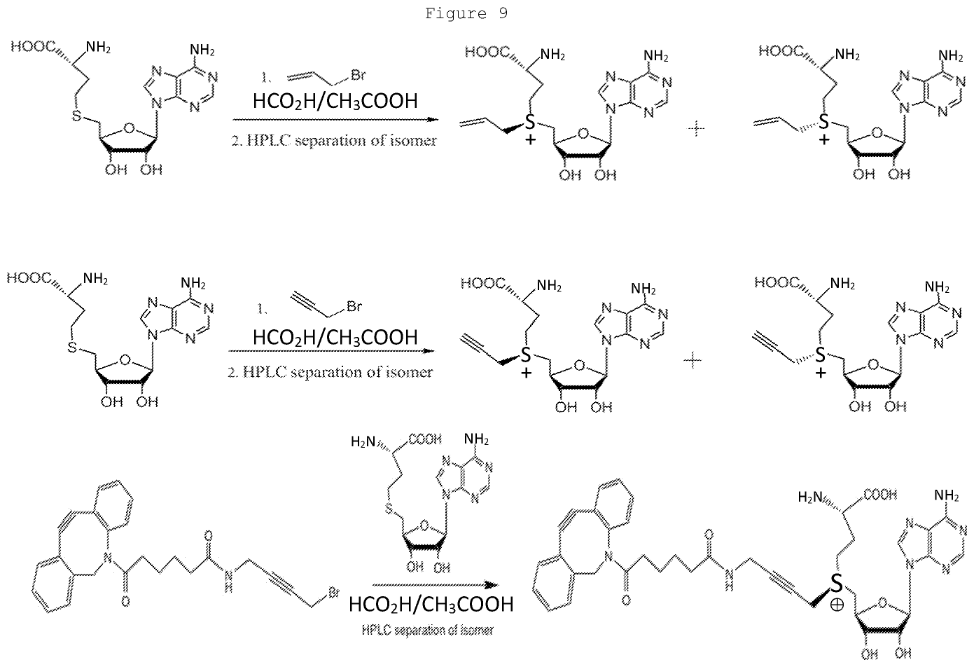

The latter is an enzyme that can convert any methylcytosine, regardless of context, to hydroxymethylcytosine. Hydroxymethyl cytosines are substrates for transfer of glucose by βGT. Thus, as shown in FIG. 1, DNA can be directly treated with βGT and UDP-glucose bearing a label that allows identification in SMS. In the case of methylated cytosines, treatment with the purified catalytic domain of TET1 to produce hydroxymethyl cytosine followed by labeled sugar transfer is carried out (Clark 2012). To prevent the possibility of TET1-mediated oxidation of 5-hmC to 5-formylC and 5-carboxylC the TET1 and βGT reactions are performed simultaneously in a single tube so as to trap 5-hmC as β-glucosyl-5 hydroxymethylcytosine before it can be further oxidized. The presence of labeled sugars will affect polymerase reaction rates much as was described for the labels in subsection 1, and to a much greater extent than simple methyl and hydroxymethyl groups. It is noted that glucosylation alone may produce a signal sufficient for accurate discrimination of modified and unmodified cytosines. However, many of the same R groups described earlier for transfer by methyltransferases can be attached to the glucose to reduce polymerase reaction rates when these are present in the template strand; as described earlier, these can include attachment of biotin for capture by streptavidin beads, cleavable linkers, etc. Examples based on the literature are shown in FIG. 10. Initial testing is performed in solution as described in subsection 1 prior to carrying out the full procedures with PacBio® and NanoSBS sequencing. An important aspect of SMS is the use of unamplified genomic DNA. Isolated single stranded DNA is circularized using adapters if desired, either with DNA Circligase (Epicentre, Inc) or by attaching dumbbell loops on both ends as in PacBio® SMRT technology, and combined with the polymerase-pore-primer complex. This entire engine is inserted into membranes on the sensor array chip and tagged nucleotides are added to accomplish the sequencing.

Summary and Conclusions

Methods for determining patterns of DNA modification have lagged far behind methods for the determination of DNA sequence. The approach presented herein is novel and is designed to have major advantages over existing methods in terms of accuracy, sensitivity, economy, and speed. The present invention is a new methylation profiling technology suited to the single molecule sequencing platforms that are approaching full maturity, and a robust system for whole genome methylation profiling.

### Example 2

In Example 1, the effect on DNA polymerase extension rates of having bulky groups attached to cytosines in the DNA template strand when using primers upstream of these positions was investigated. The template molecules used consisted of 6 CpG residues within a span of 50 bases, with the CpG cytosines being either unmodified (CpG), 5-methylcytosines (Me-CpG), or 5-octadiynecytosines (Oct-CpG). A simple fluorogenic kinetic assay was performed as shown in FIG. 4. The results show that with increased size of the bulky groups, for both Bst 2.0 polymerase and Klenow polymerase, there was a decrease in the rates of full primer extension from a t_{1/2} of 92 seconds for CpG to a t_{1/2} of 110 seconds for Me-CpG and a t_{1/2} of 138 seconds for Oct-CpG. This analysis was extended to include 5-propargyl-CpGs (Prop-CpG) at the six positions, as well as the use of templates with only three CpG, Me-CpG or Prop-CpG, spread over the same distance as the previous templates with six CpG's.

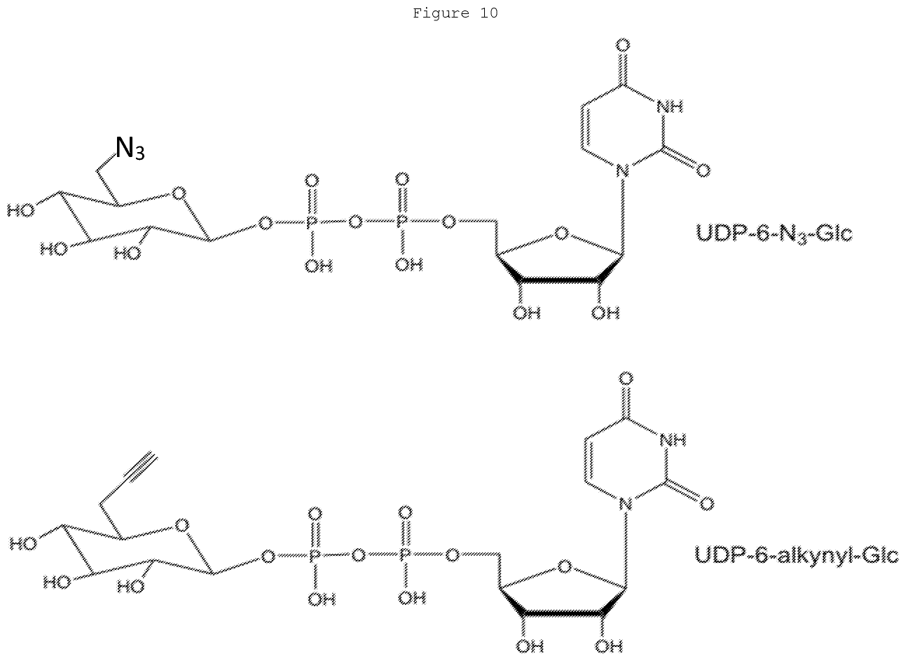

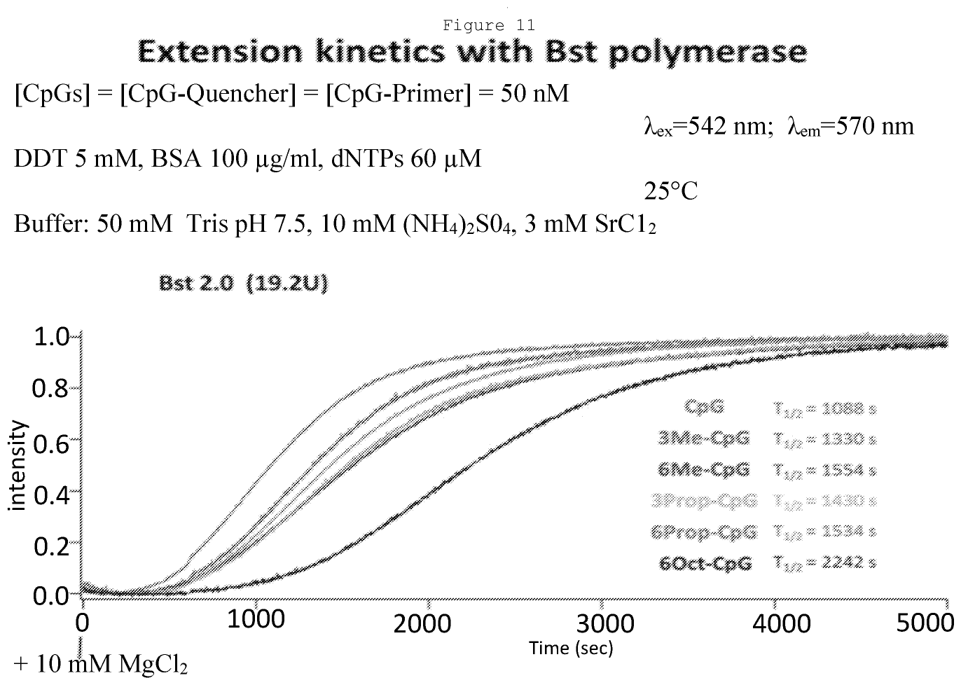

It was found that the Prop-CpG's slowed the extension to approximately the same rate as Me-CpGs with both enzymes tested (Bst 2.0 and Klenow polymerases), and the difference between the presence of three vs six modified CpG's was not significant except for the six Octadiynyl-CpG's (a template with three Octdiynyl-CpG's was not commercially available) which consistently presented with the slowest rates. Exemplary kinetic assay comparisons are presented in FIG. 11 (with Bst 2.0 polymerase) and FIG. 12 (with Klenow polymerase). These results confirm that the attachment of an octadiyne moiety to unmethylated cytosines may most easily be distinguished from unmodified and 5-methylcytosines when assessed by reaction kinetics, and therefore are ideal for the nanopore SBS approach or the PacBio® approach.

### Example 3

The enzyme-mediated modification of unmethylated CpG dinucleotides was found to be ideally suited to methylation profiling on the Oxford Nanopore MinION® sequencing platform. As discussed above, the Oxford Nanopore MinION® sequencing platform technology identifies nucleobases by measuring current blockade signals as single-stranded DNA is translocated through an alpha-hemolysin protein nanopore and thus sequencing-by-synthesis is not involved. The advantage is greatly reduced sample preparation and greatly increased throughput. The AdoMet analog preferred for use includes a propargyl group at the sulfonium. DNA will be treated with the optimized M.SssI and the propargyl analog of AdoMet so as to specifically modify all unmethylated CpG dinucleotides in each sample of DNA. The propargyl group contains a terminal alkyne that allows quick addition of essentially any azido compound via click chemistry. A variety of inexpensive and commercially available azido compounds can be covalently linked to the alkyne via click chemistry to identify and use the substituent that provides the greatest signal-to-noise ratio.

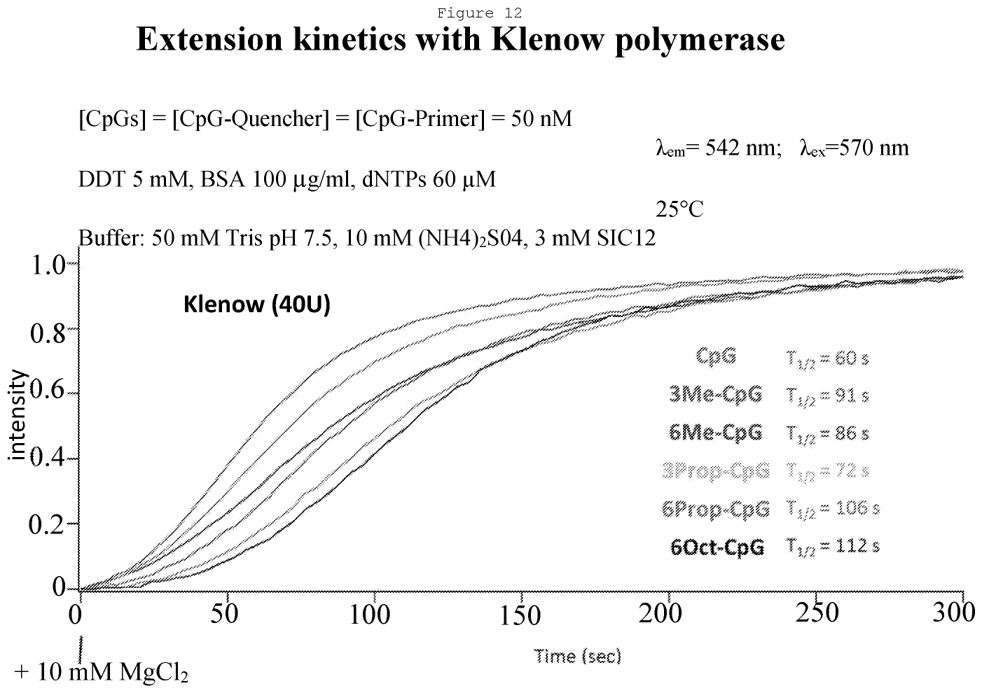

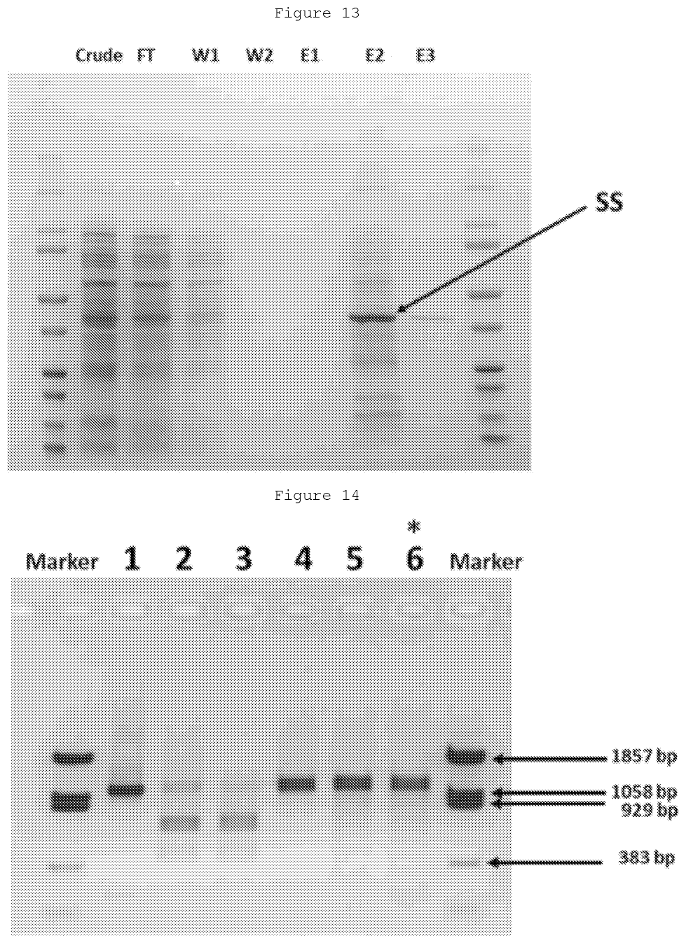

Initial tests were carried out with an ˜1.2 kb PCR product containing a HpaII cleavable CCGG, along with other CpG sites. If a methyl or other group is transferred to the 5-position of the second C in this restriction site, cleavage cannot take place. Either a wild-type M.SssI or a Q142S/N370S (SS) mutant of M.SssI is used. The latter contains a His Tag, allowing straightforward purification (FIG. 13). Next, either S-adenosylmethionine (AdoMet) or a AdoMet analogue containing a propargyl group instead of the methyl group (Prop-AdoMet) is used. As shown in FIG. 14, the wild-type enzyme can effectively transfer the methyl groups to CpG cytosines (lanes 4), and the mutant enzyme can transfer a methyl (lane 5) or propynyl group (lane 6), as assessed by the protection from cleavage by HpaII.

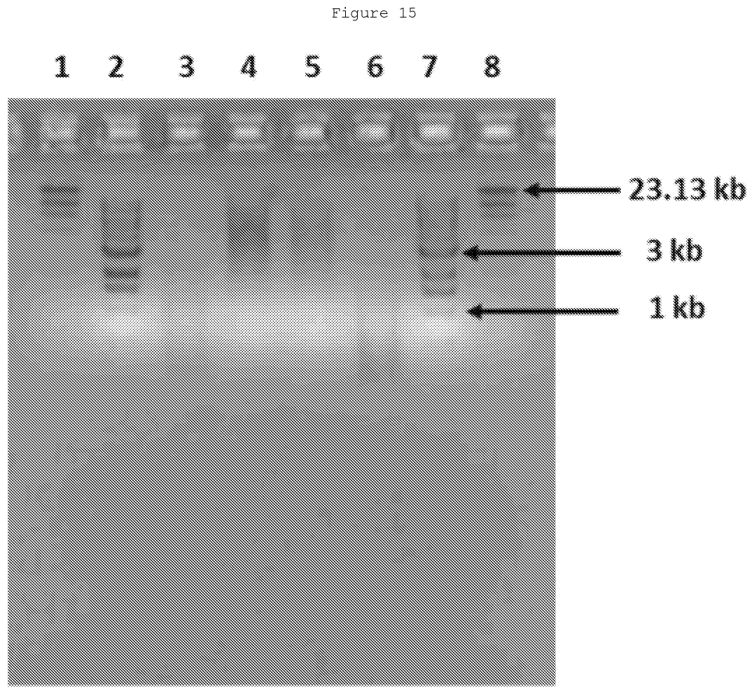

Similar assays have been carried out using E. coli whole genome DNA instead of the PCR product. The overall assay is the same except for the addition of a BamHI pre-treatment to reduce the size of the E. coli fragments, making it easier to resolve the agarose gel patterns. FIG. 15 shows initial results with AdoMet. After mutant M.SssI mediated transfer of the methyl group from AdoMet to CpG's in isolated E. coli genomic DNA, comparison of agarose gel electrophoresis patterns after treatment with HpaII should indicate the approximate percentage of CpG's that are modified by methyl transfer to the 5-position of the cytosines in these CpG's. As shown in lanes 4 and 5, near complete protection from HpaII cleavage indicates that both the wild-type and mutant enzymes are able to effectively transfer methyl groups from AdoMet to CpG cytosines.

Studies with the Prop-AdoMet analog are initiated after successful use of the assay to demonstrate transfer of AdoMet. Given the large number of CpG sites, including CCGG sites, in the E. coli genome, large amounts of Prop-AdoMet are synthesized and purified, and the ideal ratio of DNA:substrate:enzyme is found, while maintaining sufficient DNA to visualize the results by gel electrophoresis. Extra purification steps may also be necessary for the SS mutant, which displays a small amount of nuclease activity.

Actual and mock transfer samples are sent for sequencing by the Oxford Nanopore MinION® system. Because this is a single molecule approach, we are able to not only identify which specific cytosines in CpG context are available for transfer (i.e., unmethylated) but also how often they are methylated in the DNA preparations, an indication of what percentage of cells display methylation at a given CpG site.

Similar approaches are used to capture other cytosine modifications in the genome. Transfer of bulky chemical groups to 5-hydroxycytosines from UDP glucose by T4 beta-glucosyltransferases followed by sequencing, and the oxidation of 5-methylcytosines to 5-hydroxymethylcytosines, regardless of context, followed by bulky group transfer, in combination with the CpG-dependent DNA methyltransferase transfer of bulky groups, described above, reveals the modification status of cytosines throughout the genome. The same bulky group may be used for all these parallel approaches.

The methods described above are superior to all existing technologies and is very well suited to most applications, but it is not currently capable of single-cell analysis.

### REFERENCES

Clark S J, Harrison J, Paul C L, Frommer M (1994) High sensitivity mapping of methylated cytosines. Nucleic Acids Res 22(15):2990-2997.
Clark T A, Murray I A, Morgan R D, Kislyuk A O, Spittle K E, Boitano M, Fomenkov A, Roberts R J, Korlach J (2012) Characterization of DNA methyltransferase specificities using single-molecule, real-time DNA sequencing. Nucleic Acids Res 40(4):e29.
Davis B M, Chao M C, Waldor M K (2013) Entering the era of bacterial epigenomics with single molecule real time DNA sequencing. Curr Opin Microbiol 16(2):192-198.
Feng Z, Fang G, Korlach J, Clark T, Luong K, Zhang X, Wong W, Schadt E (2013) Detecting DNA modifications from SMRT sequencing data by modeling sequence context dependence of polymerase kinetics. PLoS Comput Biol 9(3):e1002935.
Flusberg B A, Webster D R, Lee J H, Travers K J, Olivares E C, Clark T A,
Korlach J, Turner S W (2010) Direct detection of DNA methylation during single-molecule, real-time sequencing. Nat Methods 7(6):461-465.
Fuller C W, Kumar S, Ju J, Davis R, Chen R (2015) Chemical methods for producing tagged nucleotides, PCT/US2015/022063, WO/2015/148402.
Fuller C W, Kumar S, Porel M, Chien M, Bibillo A, Stranges P B, Dorwart M, Tao C, Li Z, Guo W, Shi S, Korenblum D, Trans A, Aguirre A, Liu E, Harada E T, Pollard J, Bhat A, Cech C, Yang A, Arnold C, Palla M, Hovis J, Chen R, Morozova I, Kalachikov S, Russo J J, Kasianowicz J J, Davis R, Roever S, Church G M, Ju J. (2016) Real-time single-molecule electronic DNA sequencing by synthesis using polymer-tagged nucleotides on a nanopore array. Proc Natl Acad Sci USA. 113(19):5233-8. doi: 10.1073/pnas.1601782113.
Goll M G, Bestor T H (2005) Eukaryotic cytosine methyltransferases. Annu Rev Biochem 74:481-514.
Grunau C, Clark S J, Rosenthal A (2001) Bisulfite genomic sequencing: systematic investigation of critical experimental parameters. Nucleic Acids Res 29(13):E65-5.
Harrison J, Stirzaker C, Clark S J (1998) Cytosines adjacent to methylated CpG sites can be partially resistant to conversion in genomic bisulfite sequencing leading to methylation artifacts. Anal Biochem 264(1):129-32.
Kinde B, Gabel H W, Gilbert C S, Griffith E C, Greenberg M E (2015) Reading the unique DNA methylation landscape of the brain: Non-CpG methylation, hydroxymethylation, and MeCP2. Proc Natl Acad Sci USA 112(22):6800-6806.
Klimasauskas S, Kumar S, Roberts R J, Cheng X. (1994) HhaI methyltransferase flips its target base out of the DNA helix. Cell 76(2):357-69.
Klein C J, Botuvan M V, Wu Y, Ward C J, Nicholson G A, Hammans S, Hojo K, Yamanishi H, Karpf A R, Wallace D C, Simon M, Lander C, Boardman L A, Cunningham J M, Smith G E, Litchy W J, Boes B, Atkinson E J, Middha S, B Dyck P J, Parisi J E, Mer G, Smith D I, Dyck P J (2011) Mutations in DNMT1 cause hereditary sensory neuropathy with dementia and hearing loss. Nat Genet 43(6):595-600.
Kriaucionis S, Heintz N (2009) The nuclear DNA base 5-hydroxymethylcytosine is present in Purkinje neurons and the brain. Science 324(5929):929-30.
Kriukiene E, Labrie V, Khare T, Urbanviciute G, Lapinaite A, Koncevicius K, Li D, Wang T, Pai S, Ptak C, Gordevicius J, Wang S C, Petronis A, Klimasauskas S (2013) DNA unmethylome profiling by covalent capture of CpG sites. Nat Commun 4:2190. Doi: 10.1038/ncomms3190.
Kumar S, Tao C, Chien M, Hellner B, Balijepalli A, Robertson J W, Li Z, Russo J J, Reiner J E, Kasianowicz J J, Ju J (2012) PEG-labeled nucleotides and nanopore detection for single molecule DNA sequencing by synthesis. Sci Rep 2:684. Epub.
Laszlo A H, Derrington I M, Brinkerhoff H, Langford K W, Nova I C, Samson J M, Bartlett J J, Pavlenok M, Gundlach J H (2013) Detection and mapping of 5-methylcytosine and 5-hydroxymethylcytosine with nanopore MspA. Proc Natl Acad Sci USA 110(47):18904-9.
Lehman A M, du Souich C, Chai D, Eydoux P, Huang J L, Fok A K, Avila L, Swingland J, Delaney A D, McGillivray B, Goldowitz D, Argiropoulos B, Kobor M S, Boerkoel C F (2012) 19p13.2 microduplication causes a Sotos syndrome-like phenotype and alters gene expression. Clin Genet 81(1):56-63.
Li E, Bestor T H, Jaenisch R (1992) Targeted mutation of the DNA methyltransferase gene results in embryonic lethality. Cell 69(6):915-26.
Li Y, Song C-X, He C, Jin P (2012) Selective capture of 5-hydroxymethylcytosine from genomic DNA. J Vis Exp 68:4441. Online. doi:10/379/4441
Lister R, Pelizzola M, Dowen R H, Hawkins R D, Hon G, Tonti-Filippini J, Nery J R, Lee L, Ye Z, Ngo Q-M, Edsall L, Antosiewicz-Bourget J, Stewart R, Ruotti V, Millar A H, Thomson J A, Ren B, Ecker J R (2009) Human DNA methylomes at base resolution show widespread epigenomic differences. Nature 462(7271):315-322.
Lukinavicius G, Lapinaite A, Urbanaviciute G, Gerasimaite R, Klimasauskas S (2012) Engineering the DNA cytosine-5 methyltransferase reaction for sequence-specific labeling of DNA Nucleic Acids Res, 40(22), 11594-602.
G Lukinavicius, M Tomkuviene, V Masevicius, and S Klimasauskas (2013) Enhanced chemical stability of AdoMet analogues for improved methyltransferase-directed labeling of DNA. ACS Chem Biol 8:1134-1139
Nifker G, Levy-Sakin M, Berkov-Zrihen Y, Shahal T, Gabrieli T, Fridman M, Ebenstein Y (2015) One-pot chemoenzymatic cascade for labeling of the epigenetic marker 5-hydroxymethylcytosine. Chembiochem 16(13):1857-1860.
O'Donnell A H, Edwards J R, Rollins R A, Vander Kraats N D, Su T, Hibshoosh H H, Bestor T H (2014) Methylation abnormalities in mammary carcinoma: the methylation suicide hypothesis. J Cancer Ther 5(14):1311-1324.
Plongthongkum N, Diep D H, Zhang K (2014) Advances in the profiling of DNA modifications: cytosine methylation and beyond. Nat Rev Genet 15(10):647-661. PMID: 25159599.
Rand A C, Jain M, Eizenga J M, Musselman-Brown A, Olsen H E, Akeson M, Paten B (2017) Mapping DNA methylation with high-throughput nanopore sequencing. Nature Meth 14:411-413.
Renbaum P, Abrahamove D, Fainsod A, Wilson G G, Rottem S, Razin A (1990) Cloning, characterization, and expression in Escherichia coli of the gene coding for the CpG DNA methylase from Spiroplasma sp. strain MQ1(M.SssI). Nucleic Acids Res 18(5):1145-52.
Schadt E E, Banerjee O, Fang G, Feng Z, Wong W H, Zhang X, Kislyuk A, Clark T A, Luong K, Keren-Paz A, Chess A, Kumar V, Chen-Plotkin A, Sondheimer N, Korlach J, Kasarskis A (2013) Modeling kinetic rate variation in third generation DNA sequencing data to detect putative modifications to DNA bases. Genome Res 23(1):129-141.
Schreiber J, Wescoe Z L, Abu-Shumays R, Vivian J T, Baatar B, Karplus K, Akeson M (2013) Error rates for nanopore discrimination among cytosine, methylcytosine, and hydroxymethylcytosine along individual DNA strands. Proc Natl Acad Sci USA 110(47):18910-18915.
Simpson J T, Workman R E, Zuzarte M D, Dursi L J, Timp W (2017) Detecting DNA cytosine methylation using nanopore sequencing. Nature Meth 14:407-410.
Song C-X, Clark T A, Lu X-Y, Kislyuk A, Dai Q, Turner S W, He C, Korlach J (2012) Sensitive and specific single-molecule sequencing of 5-hydroxymethylcytosine. Nature Meth 9:75-77.
Stoiber M, Quick J, Egan R, Lee J E, Celniker S, Neely R K, Loman N, Pennacchio L A, Brown J (2016) De novo identification of DNA modifications enabled by genome-guided nanopore signal processing. bioRxiv doi: http://dx.doi.org/10.1101/094672.
Tahiliani M, Koh K P, Shen Y, Pastor W A, Bandukwala H, Brudno Y, Agarwal S, Iyer L M, Liu D R, Aravind L, Rao A. (2009) Conversion of 5-methylcytosine to 5-hydroxymethylcytosine in mammalian DNA by MLL partner TET1. Science. 324(5929):930-5. doi: 10.1126/science.1170116. Epub 2009 Apr. 16.
Wallace E V, Stoddart D, Heron A J, Mikhailova E, Maglia G, Donohoe T J, Bayley H (2010) Identification of epigenetic DNA modifications with a protein nanopore. Chem Commun (Camb) 46(43):8195-8197.
Warnecke P M, Stirzaker C, Song J, Grunau C, Melki J R, Clark S J (2002) Identification and resolution of artifacts in bisulfite sequencing. Methods 27(2):101-107.
Wu H Zhang Y (2015) Mechanisms and functions of Tet protein-mediated 5-methylcytosine oxidation. Genes Devel 25:2436-2452. Online: http://www.genesdev.org/cgi/dot/10.1101/gad.179184.111.
Wu H Zhang Y (2015) Charting oxidized methylcytosines at base resolution. Nat Struc Molec Biol 22(9). Published online: doi:10.1038/nsmb.3071.

What is claimed is:

1. A method of determining whether a cytosine at a predefined position within a single strand of a double-stranded DNA of known sequence is hydroxymethylated comprising:
a) contacting the double-stranded DNA with a glucosyltransferase and a uridine diphosphate glucose (UDP-glucose) so as to replace the hydrogen of hydroxymethylated cytosine with the glucose if the cytosine is hydroxymethylated; and
b) determining whether the cytosine contains the glucose;
wherein if the cytosine contains the glucose the cytosine is hydroxymethylated cytosine.

2. A method of determining whether a cytosine at a predefined position within a single strand of a double-stranded DNA of known sequence is unmethylated comprising:
a) treating the double-stranded DNA with an oxidizing agent so as to convert methylated cytosine into hydroxymethylated cytosine if cytosine is methylated;
b) contacting the treated double-stranded DNA from step a) with a glucosyltransferase and a uridine diphosphate glucose (UDP-glucose) so as to replace the hydrogen of the hydroxymethylated cytosine with the glucose if the cytosine is hydroxylated; and
c) determining whether the cytosine contains the glucose;
wherein if the cytosine does not contain glucose the cytosine is unmethylated.

3. The method of claim 2, wherein oxidizing agent is ten-eleven translocation methylcytosine dioxygenase 1 (TET1).

4. The method of any of any one of claims 1-3, wherein the glucosyltransferase is T4 β-glucosyltransferase.

5. The method of any one of claims 1-4, wherein the glucose is labeled with a detectable chemical group.

6. The method of claim 5, wherein the chemical group is selected from the group consisting of: azide, detectable alkynyl, an alkyne,

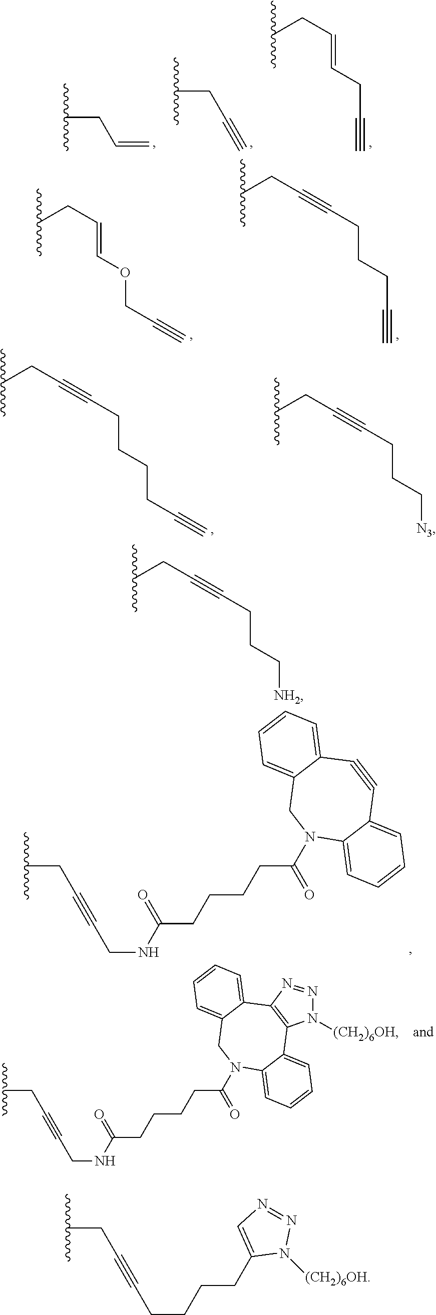

7. A method of determining whether a cytosine at a predefined position within a single strand of a double-stranded DNA of known sequence is methylated but not hydroxymethylated comprising:
a) first determining whether the cytosine is hydroxymethylated according to the method of claim 1; and
b) separately determining whether the cytosine is unmethylated according to the method of claim 2;
wherein if the cytosine is neither hydroxymethylated nor unmethylated, it is methylated.

8. A method of determining whether a cytosine present at a predefined position within a single strand of a double-stranded DNA of known sequence, and within a CpG site, is unmethylated comprising:
a) treating the double stranded DNA with a methyltransferase and an S-adenosylmethionine analog having the structure: 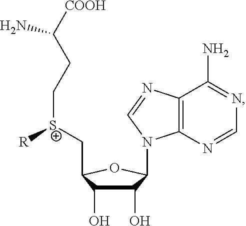
so as to replace the hydrogen attached to the 5 position of the cytosine with R if the cytosine is unmethylated and within a CpG site; and
b) determining whether the cytosine contains R;
wherein if the cytosine contains R the cytosine is a unmethylated cytosine within a CpG site,
wherein R is an octadiynyl moiety,

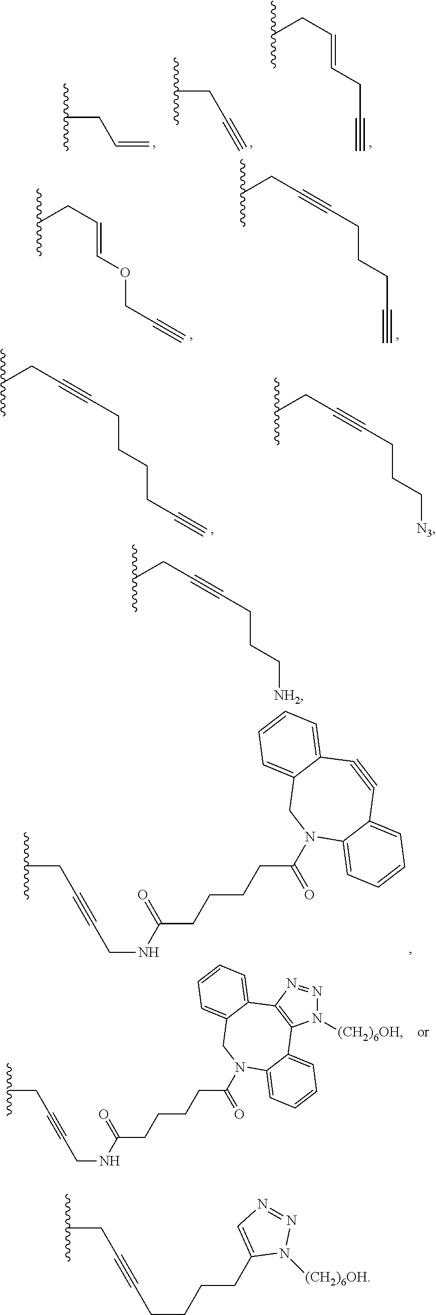

9. The method of claim 8, wherein R is a propargyl group and the method further comprises adding an azido compound to the propargyl group by click chemistry.

10. The method of claim 8 or 9, wherein the method is performed without producing (i) a U analog by photo-conversion, (ii) a thymidine analog, or (iii) a neobase.

11. The method of any one of claims 8-10, wherein the methyltransferase is a mutant M.SssI methyltransferase.

12. The method of any one of claims 8-10, wherein the methyltransferase is a mutant CpG-specific methyltransferase.

13. The method of any one of claims 8-10, wherein the methyltransferase is a C5-specific methyltransferase.

14. The method of claim 13, wherein the C5-specific methyltransferase is selected from the group consisting of M.HhaI, DNMT1, DNMT3A, DNMT3B and biologically active analogs of the foregoing.

15. A compound having the structure: 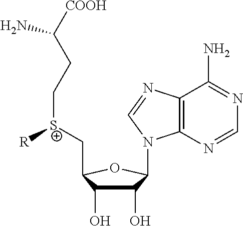
wherein R is

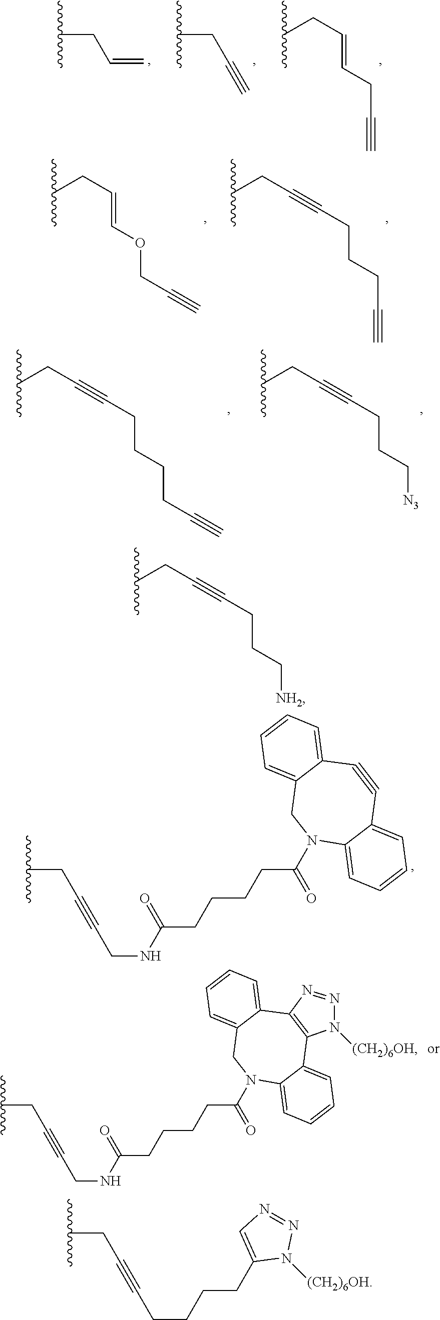

16. A composition comprising the compound of claim 15.

17. A process of preparing a derivative of a double-stranded DNA comprising contacting the double-stranded DNA with a methyltransferase and an S-adenosylmethionine analog having the structure: 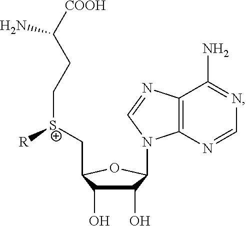
wherein R is a chemical group capable of being transferred from the S-adenosylmethionine analog by the methyltransferase to a 5 position of a non-methylated cytosine within the double-stranded DNA under conditions such that the chemical group covalently bonds to the 5 position of the non-methylated cytosine of the double-stranded DNA and thereby produces the derivative of the double-stranded DNA, wherein R has the structure:

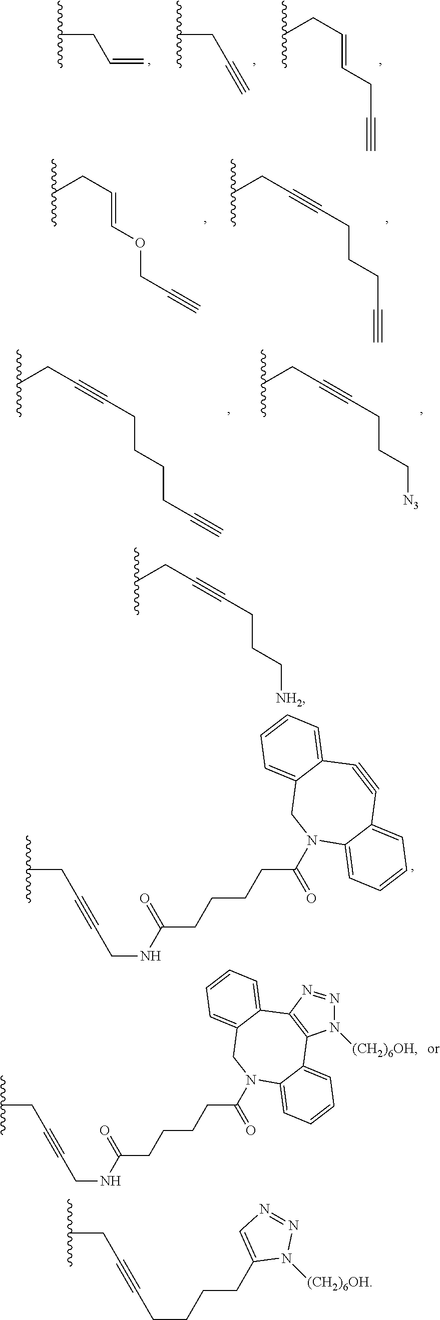

18. The process of claim 17, wherein the methyltransferase is a mutant M.SssI methyltransferase.

19. The process of claim 17, wherein the methyltransferase is a mutant CpG-specific methyltransferase.

20. The process of claim 17, wherein the methyltransferase is a C5-specific methyltransferase.

21. The process of claim 20, wherein the C5-specific methyltransferase is selected from the group consisting of M.HhaI, DNMT1, DNMT3A, DNMT3B and biologically active analogs of the foregoing.

22. A process of producing a derivative of a double-stranded DNA comprising contacting a double-stranded DNA, or a derivative thereof, with a glucosyltransferase and a uridine diphosphate glucose so as to replace the hydrogen of a hydroxymethylated cytosine with the glucose, wherein the glucose is labeled with a detectable chemical group selected from the group consisting of: an alkyne, azide, detectable alkynyl,

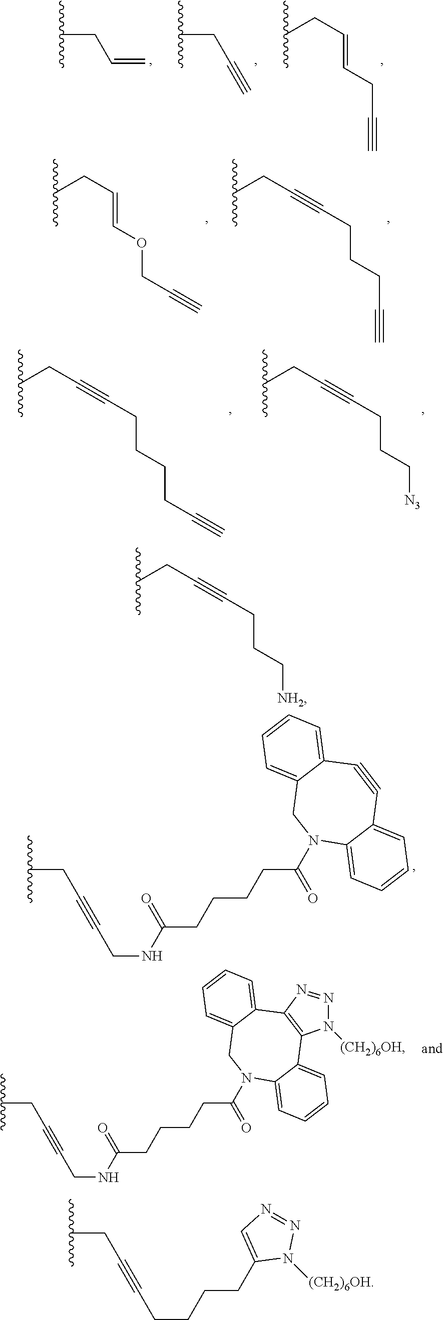

23. The process of claim 22, wherein the glucosyltransferase is T4 β-glucosyltransferase.
